(19) United States
(12) Patent Application Publication
(10) Pub. No.: US 2024/0409962 A1
(43) Pub. Date: Dec. 12, 2024

(54) ENDONUCLEASE SYSTEMS

(71) Applicant: Metagenomi, Inc., Emeryville, CA (US)

(72) Inventors: Brian C. THOMAS, Berkeley, CA (US); Christopher BROWN, Albany, CA (US); Audra DEVOTO, Emeryville, CA (US); Cristina BUTTERFIELD, Oakland, CA (US); Lisa ALEXANDER, Albany, CA (US); Daniela S.A. GOLTSMAN, Oakland, CA (US)

(21) Appl. No.: 18/660,722

(22) Filed: May 10, 2024

Related U.S. Application Data
(63) Continuation of application No. PCT/US2022/080411, filed on Nov. 23, 2022.
(60) Provisional application No. 63/283,048, filed on Nov. 24, 2021.

(51) Int. Cl. C12N 15/90; C12N 9/22; C12N 15/11

(52) U.S. Cl. CPC C12N 15/907; C12N 9/22; C12N 15/11; C12N 15/902; C12N 2310/20

(57) ABSTRACT

The present disclosure provides for endonuclease enzymes as well as methods of using such enzymes or variants thereof.

### CROSS-REFERENCE

This application is a continuation of International Application No. PCT/US2022/080411, filed on Nov. 23, 2022, which claims the benefit of U.S. Provisional Application No. 63/283,048, filed on Nov. 24, 2021, each of which is herein incorporated by reference in its entirety.

This application is related to PCT Application No. PCT/US21/24927, which is incorporated by reference herein in its entirety.

### BACKGROUND

Cas enzymes along with their associated Clustered Regularly Interspaced Short Palindromic Repeats (CRISPR) guide ribonucleic acids (RNAs) appear to be a pervasive (˜45% of bacteria, ˜84% of archaea) component of prokaryotic immune systems, serving to protect such microorganisms against non-self nucleic acids, such as infectious viruses and plasmids by CRISPR-RNA guided nucleic acid cleavage. While the deoxyribonucleic acid (DNA) elements encoding CRISPR RNA elements may be relatively conserved in structure and length, their CRISPR-associated (Cas) proteins are highly diverse, containing a wide variety of nucleic acid-interacting domains. While CRISPR DNA elements have been observed as early as 1987, the programmable endonuclease cleavage ability of CRISPR/Cas complexes has only been recognized relatively recently, leading to the use of recombinant CRISPR/Cas systems in diverse DNA manipulation and gene editing applications.

### SUMMARY

In some aspects, the present disclosure provides for an engineered nuclease system, comprising: (a) an endonuclease comprising a RuvC domain and an HNH domain, wherein the endonuclease is derived from an uncultivated microorganism; and (b) an engineered guide ribonucleic acid structure configured to form a complex with the endonuclease comprising: (i) a guide ribonucleic acid sequence configured to hybridize to a target deoxyribonucleic acid sequence; and (ii) a ribonucleic acid sequence configured to bind to the endonuclease; wherein the endonuclease has a molecular weight of about 96 kDa or less. In some embodiments, the ribonucleic acid sequence is a tracr sequence. In some embodiments, the endonuclease is an archaeal endonuclease. In some embodiments, the endonuclease is a Class 2, Type II Cas endonuclease. In some embodiments, the endonuclease comprises a sequence with at least 70%, at least 75%, at least 80% or at least 90% sequence identity to any one of SEQ ID NOs: 1 or 19-26. In some embodiments, the endonuclease further comprises an arginine-rich region comprising an RRxRR motif or a Pf14239 homology domain. In some embodiments, the arginine rich region or the Pf14239 homology domain has at least 85%, at least 90%, or at least 95% identity to an arginine rich region or Pf14239 homology domain of any one of SEQ ID NOs: 1, 19-26, or 27-28. In some embodiments, the endonuclease further comprises a REC (recognition) domain. In some embodiments, the REC domain having at least 85%, at least 90%, or at least 95% identity to a REC domain of any one of SEQ ID NOs: 1, 19-26, or 27-28.

In some aspects, the present disclosure provides for an engineered nuclease system comprising: (a) an endonuclease comprising a RuvC-I domain and an HNH domain, wherein the endonuclease is a class 2, type II Cas endonuclease; and (b) an engineered guide ribonucleic acid structure configured to form a complex with the endonuclease comprising: (i) a guide ribonucleic acid sequence configured to hybridize to a target deoxyribonucleic acid sequence; and (ii) a tracr ribonucleic acid sequence configured to bind to the endonuclease, wherein the endonuclease comprises a sequence with at least 70%, at least 75%, at least 80% or at least 90% sequence identity to any one of SEQ ID NOs: 1 or 19-26. In some embodiments, the endonuclease is an archaeal endonuclease. In some embodiments, the endonuclease is a Class 2, Type II Cas endonuclease. In some embodiments, the endonuclease further comprises an arginine-rich region comprising an RRxRR motif or a Pf14239 homology domain. In some embodiments, the arginine rich region or the Pf14239 homology domain has at least 85%, at least 90%, or at least 95% identity to an arginine rich region or Pf14239 homology domain of any one of SEQ ID NOs: 1, 19-26, or 27-28. In some embodiments, the endonuclease further comprises a REC (recognition) domain. In some embodiments, the REC domain having at least 85%, at least 90%, or at least 95% identity to a REC domain of any one of SEQ ID NOs: 1, 19-26, or 27-28. In some embodiments, the endonuclease is derived from an uncultivated microorganism. In some embodiments, the ribonucleic acid sequence comprises a sequence with at least 80% sequence identity to a natural class 2, type II tracr sequence. In some embodiments, the engineered guide ribonucleic acid structure comprises at least two ribonucleic acid polynucleotides. In some embodiments, the engineered guide ribonucleic acid structure comprises a sequence with at least 80% sequence identity a natural class 2, type II tracr sequence, wherein the engineered guide ribonucleic acid structure comprises a single ribonucleic acid polynucleotide comprising the guide ribonucleic acid sequence and the tracr ribonucleic acid sequence. In some embodiments, the guide ribonucleic acid sequence is complementary to a prokaryotic, bacterial, archaeal, eukaryotic, fungal, plant, mammalian, or human genomic sequence. In some embodiments, the guide ribonucleic acid sequence is 15-24 nucleotides in length. In some embodiments, the endonuclease comprises one or more nuclear localization sequences (NLSs) proximal to an N- or C-terminus of the endonuclease. In some embodiments, the NLS comprises a sequence selected from any one of SEQ ID NOs: 2-18. In some embodiments, the system further comprises a single- or double-stranded DNA repair template comprising from 5′ to 3′: a first homology arm comprising a sequence of at least 20 nucleotides 5′ to the target deoxyribonucleic acid sequence, a synthetic DNA sequence of at least 10 nucleotides, and a second homology arm comprising a sequence of at least 20 nucleotides 3′ to the target sequence. In some embodiments, the first or second homology arm comprises a sequence of at least 40, 80, 120, 150, 200, 300, 500, or 1,000 nucleotides. In some embodiments, the system further comprises a source of Mg²⁺. In some embodiments, the endonuclease and the tracr ribonucleic acid sequence are derived from distinct bacterial species within a same phylum In some embodiments, the endonuclease comprises a sequence with at least 70% sequence identity a natural class 2 type II tracr sequence. In some embodiments, the sequence identity is determined by a BLASTP, CLUSTALW, MUSCLE, MAFFT, or CLUSTALW with the parameters of the Smith-Waterman homology search algorithm. In some embodiments, the sequence identity is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment. In some embodiments, the endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas 13d endonuclease. In some embodiments, the endonuclease has less than 80% identity to a Cas9 endonuclease.

In some aspects, the present disclosure provides for an engineered single guide ribonucleic acid polynucleotide comprising: (a) a DNA-targeting segment comprising a nucleotide sequence that is complementary to a target sequence in a target DNA molecule; and (b) a protein-binding segment comprising two complementary stretches of nucleotides that hybridize to form a double-stranded RNA (dsRNA) duplex, wherein the two complementary stretches of nucleotides are covalently linked to one another with intervening nucleotides, and wherein the engineered guide ribonucleic acid polynucleotide is configured to form a complex with an endonuclease comprising a variant having at least 75% sequence identity to any one of SEQ ID NOs: 1 or 19-26. In some embodiments, the DNA-targeting segment is positioned 5′ of both of the two complementary stretches of nucleotides.

In some aspects, the present disclosure provides for a deoxyribonucleic acid polynucleotide encoding any of the engineered guide ribonucleic acid polynucleotides described herein.

In some aspects, the present disclosure provides for a nucleic acid comprising an engineered nucleic acid sequence optimized for expression in an organism, wherein the nucleic acid encodes a class 2, type II Cas endonuclease comprising a RuvC domain and an HNH domain, wherein the endonuclease is derived from an uncultivated microorganism, and wherein the endonuclease has a molecular weight of about 120 kDa or less, 100 kDa or less, 90 kDa or less, 60 kDa or less, or 30 kDa or less. In some embodiments, the endonuclease comprises any one of SEQ ID NOs: 1 or 19-26 or a variant thereof having at least 70% sequence identity thereto. In some embodiments, the endonuclease further comprises a sequence encoding one or more nuclear localization sequences (NLSs) proximal to an N- or C-terminus of the endonuclease. In some embodiments, the NLS comprises a sequence selected from SEQ ID NOs: 2-18. In some embodiments, the organism is prokaryotic, bacterial, eukaryotic, fungal, plant, mammalian, rodent, or human. In some embodiments, the organism is prokaryotic or bacterial, and the organism is a different organism from an organism from which the endonuclease is derived. In some embodiments, the organism is not the uncultivated microorganism.

In some aspects, the present disclosure provides for a vector comprising a nucleic acid sequence encoding a class 2, type II Cas endonuclease comprising a RuvC-I domain and an HNH domain, wherein the endonuclease is derived from an uncultivated microorganism, and wherein the endonuclease has a molecular weight of about 120 kDa or less, 100 kDa or less, 90 kDa or less, 60 kDa or less, or 30 kDa or less.

In some aspects, the present disclosure provides for a vector comprising any of the nucleic acids described herein. In some embodiments, the vector further comprises a nucleic acid encoding an engineered guide ribonucleic acid structure configured to form a complex with the endonuclease, the engineered guide ribonucleic acid structure comprising: (a) a guide ribonucleic acid sequence configured to hybridize to a target deoxyribonucleic acid sequence; and (b) a tracr ribonucleic acid sequence configured to binding to the endonuclease. In some embodiments, the vector is a plasmid, a minicircle, a CELiD, an adeno-associated virus (AAV) derived virion, or a lentivirus.

In some aspects, the present disclosure provides for a cell comprising any of the vectors described herein.

In some aspects, the present disclosure provides for a method of manufacturing an endonuclease, comprising cultivating any of the cells described herein.

In some aspects, the present disclosure provides for a method for binding, cleaving, marking, or modifying a double-stranded deoxyribonucleic acid polynucleotide, comprising: (a) contacting the double-stranded deoxyribonucleic acid polynucleotide with a class 2, type II Cas endonuclease in complex with an engineered guide ribonucleic acid structure configured to bind to the endonuclease and the double-stranded deoxyribonucleic acid polynucleotide; wherein the double-stranded deoxyribonucleic acid polynucleotide comprises a protospacer adjacent motif (PAM); wherein the endonuclease has a molecular weight of about 120 kDa or less, 100 kDa or less, 90 kDa or less, 60 kDa or less, or 30 kDa or less. In some embodiments, the endonuclease comprises a variant with at least 70%, at least 75%, at least 80%, or at least 90% sequence identity to any one of SEQ ID NOs: 1 or 19-26.

In some aspects, the present disclosure provides for a method for binding, cleaving, marking, or modifying a double-stranded deoxyribonucleic acid polynucleotide, comprising: (a) contacting the double-stranded deoxyribonucleic acid polynucleotide with a class 2, type II Cas endonuclease in complex with an engineered guide ribonucleic acid structure configured to bind to the endonuclease and the double-stranded deoxyribonucleic acid polynucleotide; wherein the double-stranded deoxyribonucleic acid polynucleotide comprises a protospacer adjacent motif (PAM); and wherein the endonuclease comprises a variant with at least 70%, at least 75%, at least 80% or at least 90% sequence identity to any one of SEQ ID NOs: 1 or 19-26. In some embodiments, the class 2, type II Cas endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas 13d endonuclease. In some embodiments, the class 2, type II Cas endonuclease is derived from an uncultivated microorganism. In some embodiments, the double-stranded deoxyribonucleic acid polynucleotide is a prokaryotic, archaeal, bacterial, eukaryotic, plant, fungal, mammalian, rodent, or human double-stranded deoxyribonucleic acid polynucleotide. In some embodiments, the double-stranded deoxyribonucleic acid polynucleotide is a prokaryotic, archaeal, or bacterial double-stranded deoxyribonucleic acid polynucleotide from a species other than a species from which the endonuclease was derived.

In some aspects, the present disclosure provides for a method of modifying a target nucleic acid locus, the method comprising delivering to the target nucleic acid locus any of the engineered nuclease systems described herein, wherein an endonuclease of the system is configured to form a complex with the engineered guide ribonucleic acid structure, and wherein the complex is configured such that upon binding of the complex to the target nucleic acid locus, the complex modifies the target nucleic locus. In some embodiments, modifying the target nucleic acid locus comprises binding, nicking, cleaving, or marking the target nucleic acid locus. In some embodiments, the target nucleic acid locus comprises deoxyribonucleic acid (DNA) or ribonucleic acid (RNA). In some embodiments, the target nucleic acid comprises genomic eukaryotic DNA, viral DNA, or bacterial DNA. In some embodiments, the target nucleic acid comprises bacterial DNA wherein the bacterial DNA is derived from a bacterial species different to a species from which the endonuclease was derived. In some embodiments, the target nucleic acid locus is in vitro. In some embodiments, the target nucleic acid locus is within a cell. In some embodiments, the endonuclease and the engineered guide nucleic acid structure are provided encoded on separate nucleic acid molecules. In some embodiments, the cell is a prokaryotic cell, a bacterial cell, a eukaryotic cell, a fungal cell, a plant cell, an animal cell, a mammalian cell, a rodent cell, a primate cell, or a human cell. In some embodiments, the cell is derived from a species different to a species from which the endonuclease was derived. In some embodiments, delivering the engineered nuclease system to the target nucleic acid locus comprises delivering any of the nucleic acids described herein or any of the vectors described herein. In some embodiments, delivering the engineered nuclease system to the target nucleic acid locus comprises delivering a nucleic acid comprising an open reading frame encoding the endonuclease. In some embodiments, the nucleic acid comprises a promoter to which the open reading frame encoding the endonuclease is operably linked. In some embodiments, delivering the engineered nuclease system to the target nucleic acid locus comprises delivering a capped mRNA containing the open reading frame encoding the endonuclease. In some embodiments, delivering the engineered nuclease system to the target nucleic acid locus comprises delivering a translated polypeptide. In some embodiments, delivering the engineered nuclease system to the target nucleic acid locus comprises delivering a deoxyribonucleic acid (DNA) encoding the engineered guide ribonucleic acid structure operably linked to a ribonucleic acid (RNA) pol III promoter. In some embodiments, the endonuclease induces a single-stranded break or a double-stranded break at or proximal to the target locus. In some embodiments, the endonuclease induces a double stranded break proximal to the target locus 5′ to a protospacer adjacent motif (PAM). In some embodiments, the endonuclease induces a double-stranded break 6-8 nucleotides or 7 nucleotides 5′ to the PAM. In some embodiments, the engineered nuclease system induces a chemical modification of a nucleotide base within or proximal to the target locus or a histone chemical modification within or proximal to the target locus. In some embodiments, the chemical modification is deamination of an adenosine or a cytosine nucleotide. In some embodiments, the endonuclease further comprises a base editor coupled to the endonuclease or a histone editor coupled to the endonuclease. In some embodiments, the base editor is an adenosine deaminase. In some embodiments, the system further comprises a base editor coupled to the endonuclease, wherein the adenosine deaminase comprises ADAR1 or ADAR2. In some embodiments, the system further comprises a base editor coupled to the endonuclease, wherein the base editor is a cytosine deaminase. In some embodiments, the cytosine deaminase comprises APOBEC1, APOBEC2, APOBEC3A, APOBEC3B, APOBEC3C, APOBEC3D, APOBEC3F, APOBEC3G, APOBEC3H, or APOBEC4.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 depicts example organizations of CRISPR/Cas loci of different classes and types.

FIG. 2 depicts a dendrogram showing homology relationships of CRISPR/Cas loci of different classes and types. Shown are a group including SMART II Cas enzyme classes (“SMART”) described herein relative to Class 2, Type II-A, II-B, and II-C Cas systems, demonstrating that these systems group into a separate class to Class 2, type II-A, II-B, and II-C Cas enzymes.

FIG. 3 shows length distribution for SMART effectors described herein, showing that SMART I and II enzymes are clustered at a lower molecular weight than Cas9-like enzymes. SMART nucleases show a bimodal distribution with one peak around 400 aa (SMART II) and a second peak around 750 aa (SMART I). Cas9 nucleases also show a bimodal distribution with peaks around 1,100 aa (e.g. SaCas9) and 1,300 aa (e.g. SpCas9).

FIG. 4 depicts an example domain organization for SMART II endonucleases (e.g. MG35 enzymes) described herein. Shown is a diagram showing the predicted domain architecture of SMART II nucleases comprising of three RuvC domains, a domain with homology to a Pfam PF14239, an HNH endonuclease domain, an unknown domain, and a recognition domain (REC).

FIG. 5A-FIG. 5B depicts the genomic context of SMART systems. FIG. 5A depicts the genomic context of the SMART II MG35-306 effector. The effector is shown as dark grey arrows, other genes are depicted as lighter grey arrows. Domains predicted for all genes in a genomic fragment are shown as grey boxes under the arrows (e.g. RRXRR protein Pfam domain). Environmental expression sequencing reads are shown aligned upstream from the effector, with paired reads connecting the putative guide RNA with the SMART II gene. FIG. 5B depicts secondary structure representation of the SMART II MG35-306 and MG35-380 putative guide RNAs.

### BRIEF DESCRIPTION OF THE SEQUENCE LISTING

The Sequence Listing filed herewith provides exemplary polynucleotide and polypeptide sequences for use in methods, compositions and systems according to the disclosure. Below are exemplary descriptions of sequences therein.

SEQ ID NO: 1, 19-26, and 27-28 show the full-length peptide sequence of a MG35 nuclease.

SEQ ID NOs: 3-18 show the sequences of example nuclear localization sequences (NLSs) that can be appended to nucleases according to the disclosure.

SEQ ID NOs: 29-30 show the nucleotide sequences of MG35 single guide RNA encoding sequences.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” generally refers to a biological cell. A cell may be the basic structural, functional or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [αS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [RI 10]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP). A nucleotide may comprise a nucleotide analog. In some embodiments, nucleotide analogs may comprise structures of natural nucleotides that are modified at any position so as to alter certain chemical properties of the nucleotide yet retain the ability of the nucleotide analog to perform its intended function (e.g. hybridization to other nucleotides in RNA or DNA). Examples of positions of the nucleotide which may be derivatized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine or guanosines, e.g., 8-bromo guanosine, 8-chloro guanosine, 8-fluoroguanosine, etc. Nucleotide analogs also include deaza nucleotides, e.g., 7-deaza-adenosine: O- and N-modified (e.g., alkylated, e.g., N6-methyl adenosine) nucleotides; and other heterocyclically modified nucleotide analogs such as those described in Herdewijn, Antisense Nucleic Acid Drug Dev., 2000 Aug. 10(4):297-310. Nucleotide analogs may also comprise modifications to the sugar portion of the nucleotides. For example the 2′ OH-group may be replaced by a group selected from H, OR, R, F, Cl, Br, I, SH, SR, NH2, NHR, NR2, COOR, or OR, wherein R is substituted or unsubstituted C1-C6 alkyl, alkenyl, alkynyl, aryl, etc. Other possible modifications include those described in U.S. Pat. Nos. 5,858,988, and 6,291,438. Examples of positions of the nucleotide which may be derivatized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine or guanosines, e.g., 8-bromo guanosine, 8-chloro guanosine, 8-fluoroguanosine, etc. Nucleotide analogs also include deaza nucleotides, e.g., 7-deaza-adenosine: O- and N-modified (e.g., alkylated, e.g., N6-methyl adenosine) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH2, NHR, NR2, COOR, or OR, wherein R is substituted or unsubstituted C1-C6 alkyl, alkenyl, alkynyl, aryl, etc. Other possible modifications include those described in U.S. Pat. Nos. 5,858,988, and 6,291,438.

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88 (which is entirely incorporated by reference herein).

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the term “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions or deletions. A non-native sequence may exhibit or encode for an activity (e.g., enzymatic activity, methyltransferase activity, acetyltransferase activity, kinase activity, ubiquitinating activity, etc.) that may also be exhibited by the nucleic acid or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid or polypeptide sequence encoding a chimeric nucleic acid or polypeptide.

The term “promoter”, as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic elements to promote transcriptional expression of an operably linked polynucleotide. Eukaryotic basal promoters often contain a TATA-box or a CAAT box.

The term “expression”, as used herein, generally refers to the process by which a nucleic acid sequence or a polynucleotide is transcribed from a DNA template (such as into mRNA or other RNA transcript) or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably generally to refer to a combination of nucleic acid sequences or elements that are expressed together or are operably linked for expression. In some cases, an expression cassette refers to the combination of regulatory elements and a gene or genes to which they are operably linked for expression.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner attributed to the full-length sequence.

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein, “synthetic” and “artificial” are used interchangeably to refer to a protein or a domain thereof that has low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains.

As used herein, the term “optimally aligned” generally refers to an alignment of two amino acid sequences that give the highest percent identity score or maximizes the number of matched residues.

The term “tracrRNA” or “tracr sequence”, as used herein, can generally refer to a nucleic acid with at least about 5%, 10%, 20%, 30%, 40%, 50%, 60%, 70%, 80%, 90%, 95%, or 100% sequence identity or sequence similarity to a wild type exemplary tracrRNA sequence (e.g., a tracrRNA from S. pyogenes S. aureus, etc.). tracrRNA can refer to a nucleic acid with at most about 5%, 10%, 20%, 30%, 40%, 50%, 60%, 70%, 80%, 90%, or 100% sequence identity or sequence similarity to a wild type exemplary tracrRNA sequence (e.g., a tracrRNA from S. pyogenes S. aureus, etc). tracrRNA may refer to a modified form of a tracrRNA that can comprise a nucleotide change such as a deletion, insertion, or substitution, variant, mutation, or chimera. A tracrRNA may refer to a nucleic acid that can be at least about 60% identical to a wild type exemplary tracrRNA (e.g., a tracrRNA from S. pyogenes S. aureus, etc.) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA (e.g., a tracrRNA from S. pyogenes S. aureus, etc) sequence over a stretch of at least 6 contiguous nucleotides. Type II tracrRNA sequences can be predicted on a genome sequence by identifying regions with complementarity to part of the repeat sequence in an adjacent CRISPR array.

As used herein, a “guide nucleic acid” can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence.” A nucleic acid-targeting segment may comprise a sub-segment that may be referred to as a “protein binding segment” or “protein binding sequence” or “Cas protein binding segment”.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with parameters of; the Smith-Waterman homology search algorithm with parameters of a match of 2, a mismatch of −1, and a gap of −1; MUSCLE with default parameters; MAFFT with parameters retree of 2 and maxiterations of 1000; Novafold with default parameters; HMMER hmmalign with default parameters.

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to any one of the endonuclease protein sequences described herein. In some embodiments, such conservatively substituted variants are functional variants. Such functional variants can encompass sequences with substitutions such that the activity of one or more critical active site residues or guide RNA binding residues of the endonuclease are not disrupted. In some embodiments, a functional variant of any of the proteins described herein lacks substitution of at least one predicted catalytic residue. In some embodiments, a functional variant of any of the proteins described herein lacks substitution of all of all predicted catalytic residues. Also provided for by the disclosure herein are altered activity variants of any of the nucleases described herein. Such altered activity variants may comprise an inactivating mutation in one or more catalytic residues or generally described for RuvC domains. Such altered activity variants may comprise a change-switch mutation in a catalytic residue of a RuvCI, RuvCII, or RuvCIII domain.

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

Included in the current disclosure are variants of any of the endonucleases described herein with sequence identity to particular domains. The domain can be an arginine rich domain, a REC (recognition) domain, a BH (bridge helix) domain, a WED (wedge) domain, a PI (PAM-interacting) domain, a PF14239 homology domain, or any other domain described herein.

As used herein, the term “RuvC_III domain” generally refers to a third discontinuous segment of a RuvC endonuclease domain (the RuvC nuclease domain being comprised of three discontiguous segments, RuvC_I, RuvC_II, and RuvC_III). A RuvC domain or segments thereof can generally be identified by alignment to documented domain sequences, structural alignment to proteins with annotated domains, or by comparison to Hidden Markov Models (HMMs) built based on documented domain sequences (e.g., Pfam HMM PF18541 for RuvC_III).

As used herein, the term “HNH domain” generally refers to an endonuclease domain having characteristic histidine and asparagine residues. An HNH domain can generally be identified by alignment to documented domain sequences, structural alignment to proteins with annotated domains, or by comparison to Hidden Markov Models (HMMs) built based on documented domain sequences (e.g., Pfam HMM PF01844 for domain HNH).

As used herein, the term “bridge helix domain” or “BH domain” generally refers to an arginine-rich helix domain present in Cas enzymes that plays an important role in initiating cleavage activity upon binding of target DNA.

As used herein, the term “recognition domain” or “REC domain” generally refers to a domain thought to interact with the repeat:anti-repeat duplex of the gRNA and to mediate the formation of a Cas endonuclease/gRNA complex.

As used herein, the term “wedge domain” or “WED domain” generally refers to a fold comprising a twisted five-stranded beta sheet flanked by four alpha helices, which is generally responsible for the recognition of the distorted repeat: anti-repeat duplex for Cas enzymes. WED domains can be responsible for the recognition of single-guide RNA scaffolds.

As used herein, the term “PAM interacting domain” or “PI domain” generally refers to a domain found in Cas enzymes positioned in the endonuclease-DNA-complex to recognize the PAM sequence on the non-complementary DNA strand of the guide RNA.

### Overview

The discovery of new Cas enzymes with unique functionality and structure may offer the potential to further disrupt deoxyribonucleic acid (DNA) editing technologies, improving speed, specificity, functionality, and ease of use. Relative to the predicted prevalence of Clustered Regularly Interspaced Short Palindromic Repeats (CRISPR) systems in microbes and the sheer diversity of microbial species, relatively few functionally characterized CRISPR/Cas enzymes exist in the literature. This is partly because a huge number of microbial species may not be readily cultivated in laboratory conditions. Metagenomic sequencing from natural environmental niches that represent large numbers of microbial species may offer the potential to drastically increase the number of new CRISPR/Cas systems documented and speed the discovery of new oligonucleotide editing functionalities. A recent example of the fruitfulness of such an approach is demonstrated by the 2016 discovery of CasX/CasY CRISPR systems from metagenomic analysis of natural microbial communities.

CRISPR/Cas systems are RNA-directed nuclease complexes that have been described to function as an adaptive immune system in microbes. In their natural context, CRISPR/Cas systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the Cas encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR-Cas systems are commonly organized into 2 classes, 5 types and 16 subtypes based on shared functional characteristics and evolutionary similarity.

Class I CRISPR-Cas systems have large, multisubunit effector complexes, and comprise Types I, III, and IV.

Type I CRISPR-Cas systems are considered of moderate complexity in terms of components. In Type I CRISPR-Cas systems, the array of RNA-targeting elements is transcribed as a long precursor crRNA (pre-crRNA) that is processed at repeat elements to liberate short, mature crRNAs that direct the nuclease complex to nucleic acid targets when they are followed by a suitable short consensus sequence called a protospacer-adjacent motif (PAM). This processing occurs via an endoribonuclease subunit (Cas6) of a large endonuclease complex called Cascade, which also comprises a nuclease (Cas3) protein component of the crRNA-directed nuclease complex. Cas I nucleases function primarily as DNA nucleases.

Type III CRISPR systems may be characterized by the presence of a central nuclease, known as Cas10, alongside a repeat-associated mysterious protein (RAMP) that comprises Csm or Cmr protein subunits. Like in Type I systems, the mature crRNA is processed from a pre-crRNA using a Cas6-like enzyme. Unlike type I and II systems, type III systems appear to target and cleave DNA-RNA duplexes (such as DNA strands being used as templates for an RNA polymerase).

Type IV CRISPR-Cas systems possess an effector complex that comprises a highly reduced large subunit nuclease (csf1), two genes for RAMP proteins of the Cas5 (csf3) and Cas7 (csf2) groups, and, in some cases, a gene for a predicted small subunit; such systems are commonly found on endogenous plasmids.

Class II CRISPR-Cas systems generally have single-polypeptide multidomain nuclease effectors, and comprise Types II, V and VI.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g. Cas9) and the repeat sequence to form a precursor dsRNA structure, which is cleaved by endogenous RNAse III to generate a mature effector enzyme loaded with both tracrRNA and crRNA. Cas II nucleases are known as DNA nucleases. Type II effectors generally exhibit a structure comprising a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand.

Type V CRISPR-Cas systems are characterized by a nuclease effector (e.g. Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, type V systems are capable of using the effector nuclease itself to cleave pre-crRNAs. Like Type-II CRISPR-Cas systems, Type V CRISPR-Cas systems are again known as DNA nucleases. Unlike Type II CRISPR-Cas systems, some Type V enzymes (e.g., Cas12a) appear to have a robust single-stranded nonspecific deoxyribonuclease activity that is activated by the first crRNA directed cleavage of a double-stranded target sequence.

Type VI CRISPR-Cas systems have RNA-guided RNA endonucleases. Instead of RuvC-like domains, the single polypeptide effector of Type VI systems (e.g. Cas13) comprises two HEPN ribonuclease domains. Differing from both Type II and V systems, Type VI systems also appear to not require a tracrRNA for processing of pre-crRNA into crRNA in some instances. Similar to type V systems, however, some Type VI systems (e.g., C2C2) appear to possess robust single-stranded nonspecific nuclease (ribonuclease) activity activated by the first crRNA directed cleavage of a target RNA.

Because of their simpler architecture, Class II CRISPR-Cas have been most widely adopted for engineering and development as designer nuclease/genome editing applications.

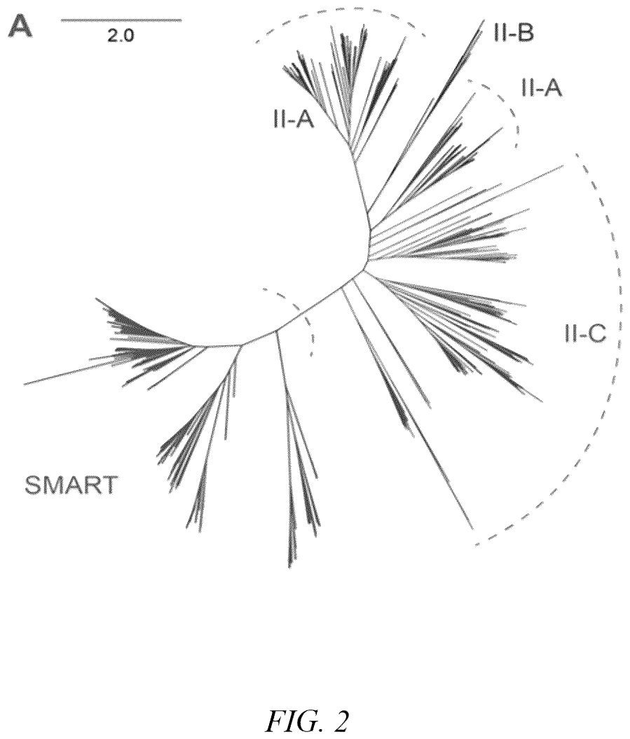

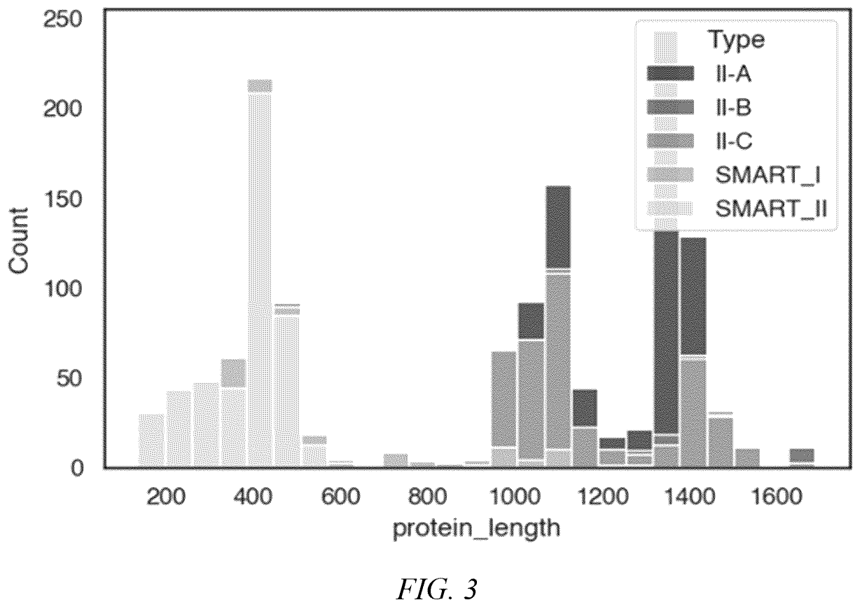

One of the early adaptations of such a system for in vitro use can be found in Jinek et al. (Science. 2012 Aug. 17; 337(6096):816-21, which is entirely incorporated herein by reference). The Jinek study first described a system that involved (i) recombinantly-expressed, purified full-length Cas9 (e.g., a Class II, Type II Cas enzyme) isolated from S. pyogenes SF370, (ii) purified mature ˜42 nt crRNA bearing a ˜20 nt 5′ sequence complementary to the target DNA sequence to be cleaved followed by a 3′ tracr-binding sequence (the whole crRNA being in vitro transcribed from a synthetic DNA template carrying a T7 promoter sequence); (iii) purified tracrRNA in vitro transcribed from a synthetic DNA template carrying a T7 promoter sequence, and (iv) Mg2+. Jinek later described an improved, engineered system wherein the crRNA of (ii) is joined to the 5′ end of (iii) by a linker (e.g., GAAA) to form a single fused synthetic guide RNA (sgRNA) capable of directing Cas9 to a target by itself (compare top and bottom panel of FIG. 2).

Mali et al. (Science. 2013 Feb. 15; 339(6121): 823-826.), which is entirely incorporated herein by reference, later adapted this system for use in mammalian cells by providing DNA vectors encoding (i) an ORF encoding codon-optimized Cas9 (e.g., a Class II, Type II Cas enzyme) under a suitable mammalian promoter with a C-terminal nuclear localization sequence (e.g., SV40 NLS) and a suitable polyadenylation signal (e.g., TK pA signal); and (ii) an ORF encoding an sgRNA (having a 5′ sequence beginning with G followed by 20 nt of a complementary targeting nucleic acid sequence joined to a 3′ tracr-binding sequence, a linker, and the tracrRNA sequence) under a suitable Polymerase III promoter (e.g., the U6 promoter).

### MG Enzymes

In one aspect, the present disclosure provides for an engineered nuclease system. The engineered nuclease system may comprise (a) an endonuclease. The endonuclease may comprise a RuvC domain and an HNH domain. The endonuclease may be derived from an uncultivated microorganism. The endonuclease may be a Cas endonuclease. The endonuclease may be a class 2 endonuclease. The endonuclease may be a class 2, type II Cas endonuclease. The engineered nuclease system may comprise an engineered guide ribonucleic acid structure. The engineered guide ribonucleic acid structure may be configured to form a complex with the endonuclease. In some cases, the engineered guide ribonucleic acid structure configured to form a complex with the endonuclease comprises a guide ribonucleic acid sequence. The guide ribonucleic acid sequence may be configured to hybridize to a target deoxyribonucleic acid sequence. In some cases, the engineered guide ribonucleic acid structure configured to form a complex with the endonuclease comprises a tracr ribonucleic acid sequence configured to bind to the endonuclease. The endonuclease may have a molecular weight of about 120 kDa or less, about 110 kDa or less, about 105 kDa or less, about 100 kDa or less, about 95 kDa or less, about 90 kDa or less, about 95 kDa or less, about 80 kDa or less, about 75 kDa or less, about 70 kDa or less, about 65 kDa or less, about 60 kDa or less, about 55 kDa or less, about 50 kDa or less, about 45 kDa or less, about 40 kDa or less, about 35 kDa or less, about 30 kDa or less, about 25 kDa or less, about 20 kDa or less, about 15 kDa or less, or about 10 kDa or less. In some cases, the endonuclease comprises a particular number of residues. The endonuclease can comprise equal to or fewer than about 1,100 residues, equal to or fewer than about 1,000 residues, equal to or fewer than about 950 residues, equal to or fewer than about 900 residues, equal to or fewer than about 850 residues, equal to or fewer than about 800 residues, equal to or fewer than about 750 residues, equal to or fewer than about 700 residues, equal to or fewer than about 650 residues, equal to or fewer than about 600 residues, equal to or fewer than about 550 residues, equal to or fewer than about 500 residues, equal to or fewer than about 450 residues, equal to or fewer than about 400 residues, or equal to or fewer than about 350 residues. The endonuclease can comprise about 700 to about 1,100 residues. The endonuclease can comprise about 400 to about 600 residues.

In some cases, the endonuclease comprises a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 1, 19-26, or 27-28.

In one aspect, the present disclosure provides for an engineered nuclease system comprising (a) an endonuclease. The endonuclease may comprise a RuvC-I domain and an HNH domain. The endonuclease may be a Cas endonuclease. The endonuclease may be a class 2 endonuclease. The endonuclease may be a class 2, type II Cas endonuclease. The engineered nuclease system may comprise (b) an engineered guide ribonucleic acid structure. The engineered guide ribonucleic acid structure may be configured to form a complex with the endonuclease. In some cases, the engineered guide ribonucleic acid structure configured to form a complex with the endonuclease comprises a guide ribonucleic acid sequence. The guide ribonucleic acid sequence may be configured to hybridize to a target deoxyribonucleic acid sequence. In some cases, the engineered guide ribonucleic acid structure configured to form a complex with the endonuclease comprises a tracr ribonucleic acid sequence configured to bind to the endonuclease. The endonuclease may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 1, 19-26, or 27-28.

In some cases, the endonuclease is derived from an uncultivated microorganism. In some cases, the tracr ribonucleic acid sequence comprises a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to at least 50, at least 60, at least 70 or at least 80 consecutive nucleotides from a natural class 2, type II tracr sequence.

The engineered guide ribonucleic acid structure may comprise at least two ribonucleic acid polynucleotides. In some cases, the engineered nuclease system described herein comprises a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to at least 50, at least 60, at least 70 or at least 80 consecutive nucleotides from a natural class 2, type II tracr sequence. The engineered guide ribonucleic acid structure may comprise a single ribonucleic acid polynucleotide. In some cases the single ribonucleic acid polynucleotide comprises the guide ribonucleic acid sequence and the tracr ribonucleic acid sequence.

In some cases, the guide ribonucleic acid sequence is complementary to a prokaryotic, bacterial, archaeal, eukaryotic, fungal, plant, mammalian, or human genomic sequence. The guide ribonucleic acid sequence may be complementary to a prokaryotic genomic sequence. The guide ribonucleic acid sequence may be complementary to a bacterial genomic sequence. The guide ribonucleic acid sequence may be complementary to a archaeal genomic sequence. The guide ribonucleic acid sequence may be complementary to a eukaryotic genomic sequence. The guide ribonucleic acid sequence may be complementary to a fungal genomic sequence. The guide ribonucleic acid sequence may be complementary to a plant genomic sequence. The guide ribonucleic acid sequence may be complementary to a mammalian genomic sequence. The guide ribonucleic acid sequence may be complementary to a human genomic sequence.

The guide ribonucleic acid sequence may be 10-30 nucleotides in length, 12-27 nucleotides in length or 15-24 nucleotides in length. The endonuclease may comprise one or more nuclear localization sequences (NLSs) proximal to an N- or C-terminus of the endonuclease. The NLS may comprise a sequence selected from SEQ ID NOs: 2-18. PGP-21 DNA M Table 1: Examples NLS Sequences that may be used with Cas effectors according to the present disclosure.

TABLE 1
Examples NLS Sequences that may be used with Cas
effectors according to the present disclosure.

| | | SEQ |
|---|---|---|
| | | ID |
| Source | NLS amino acid sequence | NO: |
| SV40 NLS | PKKKRKV | 3 |
| nucleoplasmin | KRPAATKKAGQAKKKK | 4 |
| bipartite | | |
| c-myc | PAAKRVKLD | 5 |
| c-myc | RQRRNELKRSP | 6 |
| hnRNPA1 M9 | NQSSNFGPMKGGNFGGRSSGPYGGGGQYFA | 7 |
| | KPRNQGGY | |
| Importin- | RMRIZFKNKGKDTAELRRRRVEVSVELRKA | 8 |
| alpha IBB | KKDEQILKRRNV | |
| domain | | |
| Myoma T | VSRKRPRP | 9 |
| protein | | |
| Myoma T | PPKKARED | 10 |
| protein | | |
| p53 | PQPKKKPL | 11 |
| mouse c-abl | SALIKKKKKMAP | 12 |
| IV | | |
| influenza | DRLRR | 13 |
| virus NS1 | | |
| influenza | PKQKKRK | 14 |
| virus NS1 | | |
| Hepatitis virus | RKLKKKIKKL | 15 |
| delta antigen | | |
| mouse Mx1 | REKKKFLKRR | 16 |
| protein | | |
| human | KRKGDEVDGVDEVAKKKSKK | 17 |
| poly(ADP- | | |
| ribose) | | |
| polymerase | | |
| steroid | RKCLQAGMNLEARKTKK | 18 |
| hormone | | |
| receptors | | |
| glucocorticoid | | |

The engineered nuclease system described herein may further comprise a single-stranded DNA repair template. The engineered nuclease system described herein may further comprise a double-stranded DNA repair template. The single- or double-stranded DNA repair template may comprise from 5′ to 3′: a first homology arm comprising a sequence of at least 20 nucleotides 5′ to said target deoxyribonucleic acid sequence, a synthetic DNA sequence of at least 10 nucleotides, and a second homology arm comprising a sequence of at least 20 nucleotides 3′ to said target sequence. In some cases, the first or second homology arm comprises a sequence of at least 10, 20, 30, 40, 50, 60, 70, 80, 90, 100, 110, 120, 130, 140, 150, 175, 200, 250, 300, 350, 400, 450, 500, 600, 700, 800, 900, or 1,000 nucleotides. The engineered nuclease system described herein may further comprise a source of Mg²⁺.

In some cases, the endonuclease and the tracr ribonucleic acid sequence are derived from distinct bacterial species. The distinct bacterial species may be within a same phylum. The endonuclease may comprise a sequence with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to a natural class 2, type II tracr sequence.

The sequence identity may be determined by a BLASTP, CLUSTALW, MUSCLE, MAFFT, or Smith-Waterman homology search algorithm. The sequence identity may be determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some cases, the endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas 13d endonuclease. In some cases, the endonuclease has less than 80%, less than 70%, less than 60%, less than 50%, less than 40%, less than 30%, less than 20%, or less than 10% identity to a Cas9 endonuclease.

In one aspect, the present disclosure provides an engineered single guide ribonucleic acid polynucleotide. The engineered single guide ribonucleic acid polynucleotide may comprise (a) a DNA-targeting segment. The DNA-targeting segment may comprise a nucleotide sequence. The nucleotide sequence may be complementary to a target sequence in a target DNA molecule. The engineered single guide ribonucleic acid polynucleotide may comprise (b) a protein-binding segment. The protein-binding segment may comprise two complementary stretches of nucleotides. These two complementary stretches of nucleotides may hybridize to form a double-stranded RNA (dsRNA) duplex. The two complementary stretches of nucleotides may be covalently linked to one another with intervening nucleotides. The engineered guide ribonucleic acid polynucleotide may be configured to form a complex with an endonuclease. The endonuclease may comprise a variant having at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1, 19-26, or 27-28. The DNA-targeting segment may be positioned 5′ of both of the two complementary stretches of nucleotides.

The endonuclease may comprise an arginine rich region comprising an RRxRR motif. The arginine-rich region can comprise a sequence with at least 50%, at least 55%, at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to an arginine rich region of any one of SEQ ID NOs: 1, 19-26, or 27-28. The domain boundaries of the arginine rich domain can be identified by interpro analysis (https://www.ebi.ac.uk/interpro/) or alignment to Cas9. The endo-nuclease may comprise REC domain. The REC domain can comprise a sequence with at least 50%, at least 55%, at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a REC domain of any one of SEQ ID NOs: 1, 19-26, or 27-28.

The domain boundaries of the REC domain can be identified by interpro analysis (https://www.ebi.ac.uk/interpro/) or alignment to Cas9. The endonuclease may comprise BH (Bridge Helix) domain. The BH domain can comprise a sequence with at least 50%, at least 55%, at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a BH domain of any one of SEQ ID NOs: 1, 19-26, or 27-28. The domain boundaries of the BH domain can be identified by interpro analysis (https://www.ebi.ac.uk/interpro/) or alignment to Cas9.

The endonuclease may comprise WED (wedge) domain. The WED domain can comprise a sequence with at least 50%, at least 55%, at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a WED domain of any one of SEQ ID NOs: 1, 19-26, or 27-28. The domain boundaries of the WED domain can be identified by interpro analysis (https://www.ebi.ac.uk/interpro/) or alignment to Cas9. The endonuclease may comprise PI (PAM interacting) domain. The PI domain can comprise a sequence with at least 50%, at least 55%, at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a PI domain of any one of SEQ ID NOs: SEQ ID NOs: 1, 19-26, or 27-28. The domain boundaries of the PI do-main can be identified by interpro analysis (https://www.ebi.ac.uk/interpro/) or alignment to Cas9.

In one aspect, the present disclosure provides a deoxyribonucleic acid polynucleotide encoding the engineered guide ribonucleic acid polynucleotide described herein.

In one aspect, the present disclosure provides a nucleic acid comprising an engineered nucleic acid sequence. The engineered nucleic acid sequence may be optimized for expression in an organism. The nucleic acid may encode an endonuclease. The endonuclease may be a Cas endonuclease. The endonuclease may be a class 2 endonuclease. The endonuclease may be a class 2, type II Cas endonuclease. The endonuclease may comprise a RuvC domain and an HNH domain. The endonuclease may be from an uncultivated microorganism. The endonuclease may have a molecular weight of about 120 kDa or less, 110 kDa or less, 100 kDa or less, 90 kDa or less, 80 kDa or less, 70 kDa or less, 60 kDa or less, 50 kDa or less, 40 kDa or less, 30 kDa or less, 20 kDa or less, or 10 kDa or less.

The endonuclease may comprise any one of SEQ ID NOs: 1, 19-26, or 27-28 or a variant thereof having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity thereto. The endonuclease may further comprise a sequence encoding one or more nuclear localization sequences (NLSs) proximal to an N- or C-terminus of the endonuclease. The NLS may comprise a sequence selected from SEQ ID NOs: 2-18.

The organism may be prokaryotic, bacterial, eukaryotic, fungal, plant, mammalian, rodent, or human. In some cases, the organism is prokaryotic. In some cases, the organism is prokaryotic. In some cases, the organism is bacterial. In some cases, the organism is eukaryotic. In some cases, the organism is fungal. In some cases, the organism is plant. In some cases, the organism is mammalian. In some cases, the organism is rodent. In some cases, the organism is human. If the organism is prokaryotic or bacterial, then the organism may be a different organism from an organism from which the endonuclease is derived. The organism may not be the uncultivated microorganism.

In some aspects, the present disclosure provides for an endonuclease system described herein configured to cause a chemical modification of a nucleotide base within or proximal to a target locus targeted by the endonuclease system. In this case, chemical modification of a nucleotide base generally refers to modification of the chemical moiety involved in base-pairing rather than modification of the sugar or phosphate portion of the nucleotide. The chemical modification can comprise deamination of an adenosine or a cytosine nucleotide. In some cases, endonuclease systems configured to cause a chemical modification comprises an endonuclease having a base editor coupled or fused in frame to said endonuclease. The endonuclease to which the base editor is fused or coupled can comprise a deactivating mutation in at least one catalytic residue of the endonuclease (e.g. in the RuvC domain). The base editor can be fused N- or C-terminally to said endonuclease, or linked via chemical conjugation. Base editors can include any adenosine or cytosine deaminases, including but not limited to Adenosine Deaminase RNA Specific 1 (ADAR1), Adenosine Deaminase RNA Specific 2 (ADAR2), Apolipoprotein B MRNA Editing Enzyme Catalytic Subunit 1 (APOBEC1), Apolipoprotein B MRNA Editing Enzyme Catalytic Subunit 2 (APOBEC2), Apolipoprotein B MRNA Editing Enzyme Catalytic Subunit 3A (APOBEC3A), Apolipoprotein B MRNA Editing Enzyme Catalytic Subunit 3B (APOBEC3B), Apolipoprotein B MRNA Editing Enzyme Catalytic Subunit 3C (APOBEC3C), Apolipoprotein B MRNA Editing Enzyme Catalytic Subunit 3D (APOBEC3D), Apolipoprotein B MRNA Editing Enzyme Catalytic Subunit 3F (APOBEC3F), Apolipoprotein B MRNA Editing Enzyme Catalytic Subunit 3G (APOBEC3G), Apolipoprotein B MRNA Editing Enzyme Catalytic Subunit 3H (APOBEC3H), or Apolipoprotein B MRNA Editing Enzyme Catalytic Subunit 4 (APOBEC4), or a functional fragment thereof. The base editor can comprise a yeast, eukaryotic, mammalian, or human base editor.

In some aspects, the present disclosure provides for an endonuclease system described herein configured to cause a chemical modification of histone within or proximal to a target locus targeted by the endonuclease system. In some cases, endonuclease systems configured to cause a chemical modification of a histone comprise an endonuclease having a histone editor coupled or fused in frame to said endonuclease. The histone editor can be coupled or fused N- or C-terminally to the endonuclease. In some embodiments, the chemical modification can comprise methylation, acetylation, demethylation, or deacetylation. The endonuclease to which the histone editor is fused or coupled can comprise a deactivating mutation in at least one catalytic residue of the endonuclease (e.g. in the RuvC domain). The histone editor can comprise a histone methyltransferase (e.g. ASH1L, DOT1L, EHMT1, EHMT2, EZH1, EZH2, MLL, MLL2, MLL3, MLL4, MLL5, NSD1, PRDM2, SET, SETBP1, SETD1A, SETD1B, SETD2, SETD3, SETD4, SETD5, SETD6, SETD7, SETD8, SETD9, SETDB1, SETDB2, SETMAR, SMYD1, SMYD2, SMYD3, SMYD4, SMYD5, SUV39H1, SUV39H2, SUV420H1, or SUV420H2), a histone demethylase (e.g. the KDM1, KDM2, KDM3, KDM4, KDM5, or KDM6 families), a histone acetyltransferase (e.g. GNATs or HAT family acetyltransferases), or a histone deacetylase (e.g. HDAC1, HDAC2, HDAC 3, HDAC4, HDAC5, HDAC6, HDAC7, HDAC8, HDAC9, HDAC10, HDAC11, SIRT1, SIRT2, SIRT3, SIRT4, SIRT5, SIRT6, or SIRT7). The histone editor can comprise a yeast, eukaryotic, mammalian, or human histone editor.

In one aspect, the present disclosure provides a vector comprising a nucleic acid sequence. The nucleic acid sequence may encode an endonuclease. The endonuclease may be a Cas endonuclease. The endonuclease may be a class 2 endonuclease. The endonuclease may be a class 2, type II Cas endonuclease. The endonuclease may comprise a RuvC-I domain and an HNH domain. The endonuclease may be derived from an uncultivated microorganism. The endonuclease may have a molecular weight of about 120 kDa or less, 110 kDa or less, 100 kDa or less, 90 kDa or less, 80 kDa or less, 70 kDa or less, 60 kDa or less, 50 kDa or less, 40 kDa or less, 30 kDa or less, 20 kDa or less, or 10 kDa or less.

In one aspect, the present disclosure provides a vector comprising the nucleic acid described herein. In some cases, the vector described herein further comprises a nucleic acid encoding an engineered guide ribonucleic acid structure. The engineered guide ribonucleic acid structure may be configured to form a complex with the endonuclease. The engineered guide ribonucleic acid structure may comprise (a) a guide ribonucleic acid sequence. The guide ribonucleic acid sequence may be configured to hybridize to a target deoxyribonucleic acid sequence. The engineered guide ribonucleic acid structure may comprise (b) a tracr ribonucleic acid sequence. The tracr ribonucleic acid sequence may be configured to bind to the endonuclease. In some cases, the vector is a plasmid, a minicircle, a CELiD, an adeno-associated virus (AAV) derived virion, or a lentivirus.

In one aspect, the present disclosure provides a cell comprising any of the vectors described herein.

In one aspect, the present disclosure provides a method of manufacturing an endonuclease. The method may comprise cultivating any of the cells described herein.

In one aspect, the present disclosure provides a method for binding, cleaving, marking, or modifying a double-stranded deoxyribonucleic acid polynucleotide. The method may comprise (a) contacting the double-stranded deoxyribonucleic acid polynucleotide with an endonuclease. The endonuclease may be a Cas endonuclease. The endonuclease may be a class 2 endonuclease. The endonuclease may be a class 2, type II Cas endonuclease. In some cases, the endonuclease is in complex with an engineered guide ribonucleic acid structure. The engineered guide ribonucleic acid structure may be configured to bind to the endonuclease and the double-stranded deoxyribonucleic acid polynucleotide. The double-stranded deoxyribonucleic acid polynucleotide may comprise a protospacer adjacent motif (PAM). In some cases, the endonuclease has a molecular weight of about 120 kDa or less, 110 kDa or less, 100 kDa or less, 90 kDa or less, 80 kDa or less, 70 kDa or less, 60 kDa or less, 50 kDa or less, 40 kDa or less, 30 kDa or less, 20 kDa or less, or 10 kDa or less.

The endonuclease may comprise a variant with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 1, 19-26, or 27-28.

In one aspect, the present disclosure provides a method for binding, cleaving, marking, or modifying a double-stranded deoxyribonucleic acid polynucleotide. The method may comprise contacting the double-stranded deoxyribonucleic acid polynucleotide with an endonuclease. The endonuclease may be a Cas endonuclease. The endonuclease may be a class 2 endonuclease. The endonuclease may be a class 2, type II Cas endonuclease. The endonuclease may be in complex with an engineered guide ribonucleic acid structure. The engineered guide ribonucleic acid structure may be configured to bind to the endonuclease and the double-stranded deoxyribonucleic acid polynucleotide. The double-stranded deoxyribonucleic acid polynucleotide may comprise a protospacer adjacent motif (PAM). In some cases, the endonuclease may comprise a variant with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 1, 19-26, or 27-28.

In some cases, the endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas 13d endonuclease. The endonuclease may be derived from an uncultivated microorganism.

The double-stranded deoxyribonucleic acid polynucleotide may be a prokaryotic, archaeal, bacterial, eukaryotic, plant, fungal, mammalian, rodent, or human double-stranded deoxyribonucleic acid polynucleotide. In some cases, the double-stranded deoxyribonucleic acid polynucleotide is a prokaryotic, archaeal, or bacterial double-stranded deoxyribonucleic acid polynucleotide from a species other than a species from which the endonuclease was derived.

In one aspect, the present disclosure provides a method of modifying a target nucleic acid locus. The method may comprise delivering to the target nucleic acid locus the engineered nuclease system described herein. The endonuclease may be configured to form a complex with the engineered guide ribonucleic acid structure. The complex may be configured such that upon binding of the complex to the target nucleic acid locus, the complex modifies the target nucleic locus. In some cases, modifying the target nucleic acid locus comprises binding, nicking, cleaving, or marking the target nucleic acid locus. The target nucleic acid locus comprises deoxyribonucleic acid (DNA) or ribonucleic acid (RNA). In some cases, the target nucleic acid comprises genomic eukaryotic DNA, viral DNA, or bacterial DNA.

The target nucleic acid may comprise bacterial DNA wherein the bacterial DNA is derived from a bacterial species different to a species from which the endonuclease was derived. The target nucleic acid locus may be in vitro. The target nucleic acid locus may be within a cell. The endonuclease and the engineered guide nucleic acid structure may be provided encoded on separate nucleic acid molecules. In some cases, the cell is a prokaryotic cell, a bacterial cell, a eukaryotic cell, a fungal cell, a plant cell, an animal cell, a mammalian cell, a rodent cell, a primate cell, or a human cell. In some cases, the cell is derived from a species different to a species from which the endonuclease was derived.

The method described herein may comprise delivering the engineered nuclease system to the target nucleic acid locus which further comprises delivering the nucleic acid described herein or the vector described herein. The method described herein may comprise delivering the engineered nuclease system to the target nucleic acid locus which further comprises delivering a nucleic acid comprising an open reading frame encoding the endonuclease. The nucleic acid may comprise a promoter to which the open reading frame encoding the endonuclease is operably linked.

The method described herein may comprise delivering the engineered nuclease system to the target nucleic acid locus which comprises delivering a capped mRNA containing the open reading frame encoding the endonuclease. In some cases, delivering the engineered nuclease system to the target nucleic acid locus comprises delivering a translated polypeptide.

The method described herein may comprise delivering the engineered nuclease system to the target nucleic acid locus which comprises delivering a deoxyribonucleic acid (DNA) encoding the engineered guide ribonucleic acid structure operably linked to a ribonucleic acid (RNA) pol III promoter. In some cases, the endonuclease induces a single-stranded break or a double-stranded break at or proximal to the target locus.

Systems of the present disclosure may be used for various applications, such as, for example, nucleic acid editing (e.g., gene editing), binding to a nucleic acid molecule (e.g., sequence-specific binding). Such systems may be used, for example, for addressing (e.g., removing or replacing) a genetically inherited mutation that may cause a disease in a subject, inactivating a gene in order to ascertain its function in a cell, as a diagnostic tool to detect disease-causing genetic elements (e.g. via cleavage of reverse-transcribed viral RNA or an amplified DNA sequence encoding a disease-causing mutation), as deactivated enzymes in combination with a probe to target and detect a specific nucleotide sequence (e.g. sequence encoding antibiotic resistance int bacteria), to render viruses inactive or incapable of infecting host cells by targeting viral genomes, to add genes or amend metabolic pathways to engineer organisms to produce valuable small molecules, macromolecules, or secondary metabolites, to establish a gene drive element for evolutionary selection, to detect cell perturbations by foreign small molecules and nucleotides as a biosensor.

TABLE 2
Sequence Listing of protein and nucleic acid sequences referred to herein

| | SEQ | | | | | |
|---|---|---|---|---|---|---|
| | ID | | | | Other | |
| Category | NO: | Description | Type | Organism | information | Sequence |
| MG35 | 1 | MG35-145 | Protein | Unknown | Unculti- | SVLSTDAVVPLLITATGH |
| effectors | | effector | | | vated | GNRQMCGTNKYGFPSR |
| | | | | | organism | HRQRQKHHYGYQTGDM |
| | | | | | | VRAVVTSGKKVGKYVG |
| | | | | | | RVLTRATGSFDIRTKQG |
| | | | | | | RVQGISHRFCTPVHRLD |
| | | | | | | GYSYQKGEVA |
| target | 2 | test target | Nucleotide | Artificial | | CGUGAGCCACCACGUC |
| | | sequence | | Sequence | | GCAAGCCUCGAC |
| NLS | 3 | SV40 NLS | protein | Simian | | PKKKRKV |
| | | | | vacuola- | | |
| | | | | ting virus | | |
| | | | | 40 T | | |
| NLS | 4 | nucleoplasmin | protein | Human | | KRPAATKKAGQAKKKK |
| | | bipartite | | | | |
| | | NLS | | | | |
| NLS | 5 | c-myc NLS | protein | Human | | PAAKRVKLD |
| NLS | 6 | c-myc NLS | protein | Human | | RQRRNELKRSP |
| NLS | 7 | hnRNPA1 | protein | Mouse | | NQSSNFGPMKGGNFGG |
| | | M9 NLS | | | | RSSGPYGGGGQYFAKPR |
| | | | | | | NQGGY |
| NLS | 8 | Importin- | protein | Human | | RMRIZFKNKGKDTAELR |
| | | alpha IBB | | | | RRRVEVSVELRKAKKDE |
| | | domain | | | | QILKRRNV |
| | | NLS | | | | |
| NLS | 9 | Myoma T | protein | Murine | | VSRKRPRP |
| | | protein | | polyomav | | |
| | | NLS | | irus | | |
| NLS | 10 | Myoma T | protein | Murine | | PPKKARED |
| | | protein | | polyomav | | |
| | | NLS | | irus | | |
| NLS | 11 | p53 NLS | protein | Human | | PQPKKKPL |
| NLS | 12 | mouse c-abl | protein | Mouse | | SALIKKKKKMAP |
| | | IV NLS | | | | |
| NLS | 13 | influenza | protein | Influenza | | DRLRR |
| | | virus NS1 | | virus NS1 | | |
| | | NLS | | | | |
| NLS | 14 | influenza | protein | Influenza | | PKQKKRK |
| | | virus NS1 | | virus NS1 | | |
| | | NLS | | | | |
| NLS | 15 | Hepatitis | protein | Hepatitis | | RKLKKKIKKL |
| | | virus delta | | virus | | |
| | | antigen | | delta | | |
| | | NLS | | | | |
| NLS | 16 | mouse Mx1 | protein | Mouse | | REKKKFLKRR |
| | | protein | | | | |
| NLS | 17 | human | protein | Human | | KRKGDEVDGVDEVAKK |
| | | poly(ADP- | | | | KSKK |
| | | ribose) | | | | |
| | | polymerase | | | | |
| | | NLS | | | | |
| NLS | 18 | steroid | protein | Human | | RKCLQAGMNLEARKTK |
| | | hormone | | | | K |
| | | receptors | | | | |
| | | glucocorticoid | | | | |
| | | NLS | | | | |
| MG35 | 19 | MG35-182 | protein | unknown | unculti- | APITAISQELVRFDMQAI |
| effectors | | effector | | | vated | ENPEIAGVGYQQGELAG |
| | | | | | organism | YEVNDYLLEKWNRTCA |
| | | | | | | YCGARDVPLQIEHITPRS |
| | | | | | | RGGSNRVSNLAIACEPC |
| | | | | | | NQDKDNQPVAVFLAAR |
| | | | | | | PALLKRITAQAKAPLKD |
| | | | | | | AAAVNATRWALFNVLQ |
| | | | | | | ATGLPVETGSGGRTKYN |
| | | | | | | RARLGIPKAHALDAACV |
| | | | | | | GQVDTLHGWRCPTLAIK |
| | | | | | | ATGRGAYQRTRLDRFGF |
| | | | | | | PRGYLTRHKRFHGFQTG |
| | | | | | | DRVVAQVPTGKKAGCH |
| | | | | | | VGRVAVRATGSFNIQTI |
| | | | | | | GGVVQGISHRYCRVLQR |
| | | | | | | ADGYGYSLNPKSTEVGT |
| | | | | | | HHAP |
| MG35 | 20 | MG35-306 | protein | unknown | unculti- | MNRNSVFVLDTNKKPC |
| effectors | | effector | | | vated | NPVHPAVARKLLKQKK |
| | | | | | organism | AAVFRRYPFTIILKEESK |
| | | | | | | DETKELRVKIDPGAKTT |
| | | | | | | GLAIASETNIVWCADLE |
| | | | | | | HRGFQIRDNLSNRKIRRR |
| | | | | | | SRRNRKTRYRKPRFLNR |
| | | | | | | KRPKGWLPPSLMSRVFN |
| | | | | | | IESWVNKLCRLARITAIS |
| | | | | | | QELVRFDTQKMVNPEIN |
| | | | | | | GTEYQRGELFGYEVREY |
| | | | | | | LLDKFNRTCVYCNTKEG |
| | | | | | | PFNLDHFHPKSKGGSDR |
| | | | | | | VSNLVLSCVKCNQKKD |
| | | | | | | NQLPADFLSDKALLLAK |
| | | | | | | IEKQRKQPLADTVAVNA |
| | | | | | | TRWKLKEVLENTGLPVE |
| | | | | | | VGSGGLTKFNRRKLGIS |
| | | | | | | KSHWTDAACVGTSTPNS |
| | | | | | | LNVKGYQPLLIKAMGRG |
| | | | | | | SRHKVNSDKYGFPRGAP |
| | | | | | | KFRQKSFFGFQTGDMVK |
| | | | | | | AIVPKGKYTGTHTGRIA |
| | | | | | | VRKKGNFKIKTSTQTFD |
| | | | | | | VNHKYCQHIHKSDGFAY |
| | | | | | | SFGELVKQKVKVVKPTI |
| | | | | | | NKPITPTQLNLFDTTEFS |
| | | | | | | TQTIQTKIKRTHKSKGTE |
| | | | | | | GEQLSLF |
| MG35 | 21 | MG35-332 | protein | unknown | unculti- | MRSQFLALSFAIKQTYR |
| effectors | | effector | | | vated | VEVSVVNPKSFRNIAKA |
| | | | | | organism | NITRKETQALSKVFVLD |
| | | | | | | TNKQPLNPIHSGRARML |
| | | | | | | LSQEKSAVFRRYPFTVIL |
| | | | | | | NKEVVNPKVESLRVKID |
| | | | | | | PGAKTTGLGLVNDSTGE |
| | | | | | | VVWAAQLQHRGFQIRD |
| | | | | | | ALTSRRQLRRSRRNRKT |
| | | | | | | RYRKSRFLNRTRPEGWL |
| | | | | | | PPSLNSRVANILTWVNR |
| | | | | | | LSSISQITAISQELVRFDT |
| | | | | | | QQMEDAEISGIGYQQGT |
| | | | | | | LFGYEIREFLLEKWNRT |
| | | | | | | CAYCGEKDTQLEIEHIN |
| | | | | | | ARSKGGSNRISNLCLAC |
| | | | | | | TSCNQKKGNQDIKDFLK |
| | | | | | | GKPDLLKCILSQAKRPL |
| | | | | | | ADTAAVNATRWNLYYN |
| | | | | | | LKKTGLPVEVGTGGRTK |
| | | | | | | YNRTIRGLDKTHYWDA |
| | | | | | | ACVGASTPEQLITSEVKP |
| | | | | | | LLIAAKGHGTRQQCRTD |
| | | | | | | RFGFPSRYCKRTKFHKG |
| | | | | | | FQTGDIVKAVVTSGKKI |
| | | | | | | GTYVGRVATRATGSFNI |
| | | | | | | STTDGLVQGISHKYCLPL |
| | | | | | | HKKDGYSYIQ |
| MG35 | 22 | MG35-380 | protein | unknown | unculti- | MVFVLDRRKKPLMPCS |
| effectors | | effector | | | vated | AKRARLLLAQGRARVH |
| | | | | | organism | RLVPFTIRLVDRTVEDSS |
| | | | | | | LQPVRVKIDPGSKTTGM |
| | | | | | | ALVRDEVAVDPETGEIR |
| | | | | | | RVAHGLFLAELHHRGH |
| | | | | | | AIRDALAARRAFRRRRR |
| | | | | | | AQLRHRAP |
| MG35 | 23 | MG35-381 | protein | unknown | unculti- | MVFVLDRRKKPLMPCS |
| effectors | | effector | | | vated | AKRARLLLAQGRARVH |
| | | | | | organism | RLVPFTIRLVDRTVGDSS |
| | | | | | | LQPVRVKIDPGSKTTGM |
| | | | | | | ALVRDEVAVD |
| MG35 | 24 | MG35-382 | protein | unknown | unculti- | MVFVLDRRKKPLMPCS |
| effectors | | effector | | | vated | AKRARLLLAQGRARVH |
| | | | | | organism | RLVPFTIRLVDRTVGDSS |
| | | | | | | LQPVRVKIDPGSKTTGM |
| | | | | | | ALVRDEVAVDPETGEIR |
| | | | | | | RVAHGLFLAELHHRGH |
| | | | | | | AIRDALAARRAFRRRRR |
| | | | | | | AQLRHRAPRFDNRRRPD |
| | | | | | | GWLAPSLQHRVDTTMA |
| | | | | | | WVRRFQRCAPMTALSQ |
| | | | | | | ELVRFDTQLMQNPEISG |
| | | | | | | VAYQQGTLAGYEMREY |
| | | | | | | LLEKWGRRCAYCPATD |
| | | | | | | VPLEVEHIVPRSRGGSNR |
| | | | | | | IANLTLACRTCNQAKDN |
| | | | | | | RDITEFLAHDPVRLARIQ |
| | | | | | | AQAKAPLRDAAAVNST |
| | | | | | | RWALIQALKATALPVET |
| | | | | | | GTGGRTKFNRARLHLPK |
| | | | | | | THALDAACVGEVDAVL |
| | | | | | | AADKPALCIRATGRGSY |
| | | | | | | QRTRLDRFGFPRGVLTR |
| | | | | | | RKHHFGFQTGDMVRAA |
| | | | | | | VPSGKKAGTYTGRVAV |
| | | | | | | RASGSFNIQTPQGVIQGV |
| | | | | | | SHRHCRMLQRADGYGY |
| | | | | | | SWTKIVSQQGVAGTGA |
| | | | | | | AQAAALSLSGLNAGVSR |
| | | | | | | AN |
| MG35 | 25 | MG35-383 | protein | unknown | unculti- | MVFVLDRRKKPLMPCS |
| effectors | | effector | | | vated | AKRARLLLAQGRARVH |
| | | | | | organism | RLVPFTIRLVDRTVGDSS |
| | | | | | | LQPVRVKIDPGSKTTGM |
| | | | | | | ALVRDEVAVDPETGEIR |
| | | | | | | RVAHGLFLAELHHRGH |
| | | | | | | AIRDALAARRAFRRRRR |
| | | | | | | AQLRHRAPRFDNRRRPD |
| | | | | | | GWLAPSLQHRVDTTMA |
| | | | | | | WVRRFQRCAPMTALSQ |
| | | | | | | ELVRFDTQLMQNPEISG |
| | | | | | | VAYQQGTLAGYEMREY |
| | | | | | | LLEKWGRRCAYCPATD |
| | | | | | | VPLEVEHIVPRSRGGSNR |
| | | | | | | IATPPLACRTCTQAKDN |
| | | | | | | RAITEFLAHDPVRLARIQ |
| | | | | | | AQAKAPLRDAAAVNST |
| | | | | | | RWALIQALKATALPVET |
| MG35 | 26 | MG35-407 | protein | unknown | unculti- | TALSQELVRFDTQLM |
| effectors | | effector | | | vated | QNPEISGVAYQQGTLA |
| | | | | | organism | GYEMREYLLEKWGRR |
| | | | | | | CAYCPATDVPLEVEHI |
| | | | | | | VPRSRGGSNRIANLTL |
| | | | | | | ACRTCNQAKDNRDIT |
| | | | | | | EFLAHDPVRLARIQAQ |
| | | | | | | AKAPLRDAAAVNSTR |
| | | | | | | WALIQALKATALPVE |
| | | | | | | TGTGGRTKFNRARLH |
| | | | | | | LPKTHALDAACVGEV |
| | | | | | | DAVLAADKPALCIRA |
| | | | | | | TGRGSYQRTRLDRFGF |
| | | | | | | PRGVLTRRKHHFGFQ |
| | | | | | | TGDMVRAAVPSGKKA |
| | | | | | | GTYTGRVAVRASGSF |
| | | | | | | NIQTPQGVIQGVSHRH |
| | | | | | | CRMLQRADGYGYSW |
| | | | | | | TKIVSQQGVAGTGAA |
| | | | | | | QAAALSLSGLNAGVS |
| | | | | | | RAN |
| MG102 | 27 | MG102-26 | proein | unknown | unculti- | NSWTYIRNNLESVIKR |
| effectors | | effector | | | vated | QTGRNRYCRKHSEMY |
| | | | | | organism | VNHILNGKPIPFKTTLT |
| | | | | | | EKGIVSKREEILFQKL |
| | | | | | | WRYIEAIVLPLAPDGI |
| | | | | | | DRVVVERTAFDLLAG |
| | | | | | | TRKQRDKLSDQALEE |
| | | | | | | MYQSGPRLGFKNDRE |
| | | | | | | MLKAEFGGLCAYCGN |
| | | | | | | PAESIFDREHILPRSDF |
| | | | | | | FFDSYINIVPACPTCNR |
| | | | | | | NLKGKRPLSASGLRIP |
| | | | | | | DKAYEAYSNYLKSKY |
| | | | | | | KQKPPHLFHTIKKGIL |
| | | | | | | KLMRQPDRMWEAEE |
| | | | | | | YLILISKQFTEIVQTQR |
| | | | | | | GPRSLARYLSERLRRH |
| | | | | | | QGRIPNVSFFNGRHTA |
| | | | | | | IWRQAAYPDFNKISDK |
| | | | | | | FEGGVVNHAIDALLL |
| | | | | | | ACKLPSVSKLEAKRLQ |
| | | | | | | INDLSEWKRSVRRLAP |
| | | | | | | APGPNGIPDLPIPRDIV |
| | | | | | | PGFEKVENDNYISADL |
| | | | | | | AM |
| MG33 | 28 | MG33-25 | protein | unknown | unculti- | RQIKKNELKEIVSAFG |
| effectors | | effector | | | vated | RKHHLPRAATEQWEG |
| | | | | | organism | ELAGLLDKILRPARFD |
| | | | | | | NRLRSGCSWCGKKTP |
| | | | | | | RLIKREVREMALRAA |
| | | | | | | VENIRVKDGGRLRPLK |
| | | | | | | QEEKEPFLSWWNKRG |
| | | | | | | EKFQFPKGAQIAVTER |
| | | | | | | APTPGNIAKYLEQIGA |
| | | | | | | VKSWARNAKGKGKY |
| | | | | | | DFPMLPQLNDLLNRTP |
| | | | | | | KQGRSKLCIQHLRMA |
| | | | | | | GQGGFLCNRHVNTVC |
| | | | | | | RLNTDGTHTKLDGLT |
| | | | | | | MPVGRYNAPNPKREQ |
| | | | | | | HDSRVLNRLESILFIKG |
| | | | | | | KRGKNAWRHGPVSF |
| | | | | | | MTLEIPDPQTDRVGKK |
| | | | | | | QQTERQDRTLHQRIHE |
| | | | | | | ETKGACVYCGNPVSP |
| | | | | | | ENMEMEHIVPKAKNG |
| | | | | | | PDIRTNRVASCRQCNH |
| | | | | | | PDTGKGGRLPSEWLT |
| | | | | | | GQDWKDFENRVKAFR |
| | | | | | | LPEFKEKLLLLPKGES |
| | | | | | | FPEDPTSLARVGARTG |
| | | | | | | AFIRQVQELFKRYGVE |
| | | | | | | SPGLTYTPGKSHIQVV |
| | | | | | | RGRDTRTLRTSWLFK |
| | | | | | | DKKEKEKNFPEKDRT |
| | | | | | | DLFNHAQDAALLAAV |
| | | | | | | PPHT |

### EXAMPLES

### Example 1—Discovery of New Cas Effectors by Metagenomics

Metagenomic samples were collected from sediment, soil and animal. Deoxyribonucleic acid (DNA) was extracted with a Zymobiomics DNA mini-prep kit and sequenced on an Illumina HiSeq® 2500. Samples were collected with consent of property owners. DNA was extracted from samples using either the Qiagen DNeasy PowerSoil Kit or the ZymoBIOMICS DNA Miniprep Kit. DNA was sent for sequencing library preparation (Illumina TruSeq) and sequencing on an Illumina HiSeq 4000 or Novaseq to the Vincent J. Coates Genomics Sequencing Laboratory at UC Berkeley (paired 150 base pair (bp) reads with a 400-800 bp target insert size). Additionally, publicly available high temperature, as well as soil and ocean metagenomic sequencing data were downloaded from the NCBI SRA. Sequencing reads were trimmed using BBMap (Bushnell B., sourceforge.net/projects/bbmap/) and assembled with Megahit. Protein sequences were predicted with Prodigal. HMM profiles of documented Type II CRISPR nucleases were built and searched against all predicted proteins using HMMER3 (hmmer.org). CRISPR arrays were predicted on assembled contigs with Minced (https://github.com/ctSkennerton/minced). Taxonomy was assigned to proteins with Kaiju and contig taxonomy was determined by finding the consensus of all encoded proteins.

Predicted and reference (e.g. SpCas9, SaCas9, and AsCas9) Type II effector proteins were aligned with MAFFT and phylogenetic trees were inferred using FastTree2. Novel families were identified from clades composed of sequences recovered from this study. From within families, candidates were selected if they contained all components facilitating laboratory analysis (i.e. they were found on a well-assembled and annotated contig with a CRISPR array). Selected representative and reference sequences were aligned using MUSCLE to identify catalytic and PAM interacting residues. This metagenomic workflow resulted in the delineation of the SMART (SMall ARchaeal-associaTed) MG35 family endonuclease systems described herein.

### Discovery of SMART Endonucleases Containing Active Residue Signatures

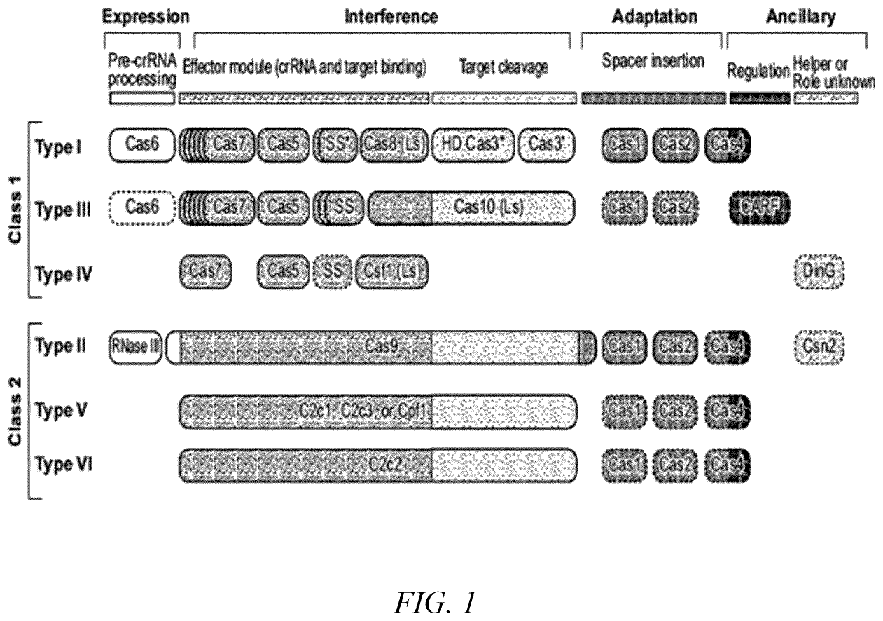

Mining of tens of thousands of high quality CRISPR Cas systems assembled from metagenomic data uncovered novel effectors containing both RuvC and HNH domains, but that were of unusually small size (<900 aa). These effector nucleases showed only low sequence similarity (<20% amino acid identity) to archaeal Cas9 endonucleases as a reference point. Phylogenetic analysis of effector protein sequences indicated that the SMART systems are a divergent group relative to well-studied Type II systems from subtype A, B, or C (FIG. 1).

These compact “SMART” effectors (˜400-1000 amino acids, FIG. 2) appeared in loci in the genome adjacent to CRISPR arrays.

### Multiple Groups of SMART Endonucleases

Based on the location of important catalytic and binding residues, SMART nucleases comprise three RuvC domains, an arginine rich region usually containing an RRxRR motif, an HNH endonuclease domain, and a putative recognition domain. Given differences in SMART effector size, phylogenetic relationship, and both operon and domain architecture, we currently classified these systems into two primary groups: SMART I and SMART II.

The salient features of SMART II enzymes (e.g. the MG35 family enzymes identified herein) is outlined in Table 3 below, which also illustrates differences compared to Class 2, Type II A/B/C Cas enzymes.

TABLE 3
Attributes of SMART II group enzymes described herein

| Attribute | SMART II | Type II: A, B, C |
|---|---|---|
| Zn-binding residues | yes | no |
| Bridge helix | no | yes |
| PAM interacting | no | yes |
| and WED domain | | |
| RRxRR motif | yes | no |
| REC domain | Novel domain at C- | Cas9 REC domain |
| | terminus | |
| Domain w/ | yes | no |
| PF14239 homology | | |
| Monophyletic clade | no | yes |
| Related to TnpB | yes | yes |
| Operon contains | sometimes | no |
| IS605 Tns repeats | | |
| <900 aa | yes | no |
| CRISPR-associated | sometimes | yes |
| Contains RuvC and | yes | yes |
| HNH | | |

### SMART II Endonucleases

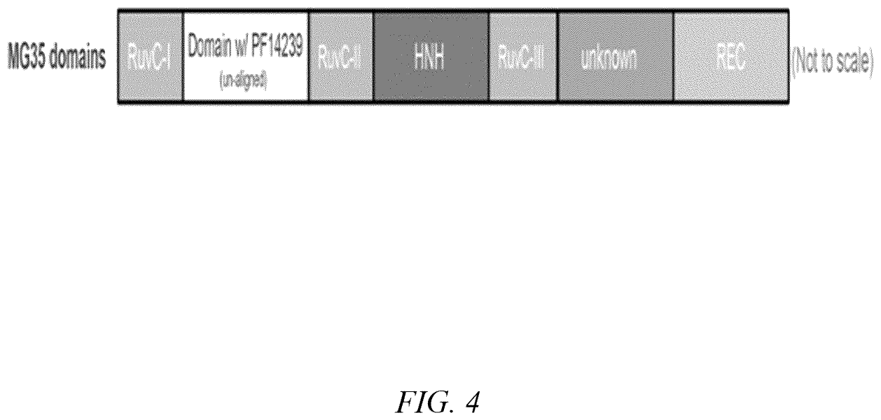

SMART II effectors (an example domain organization of which is shown in FIG. 4) have a size distribution that skews smaller (˜400 amino acids-600 amino acids) vs. SMART I effectors. Their genomic context suggested unusual repetitive regions or CRISPR arrays. The non-CRISPR repetitive regions contain direct repeats that range in size from about 10 to over 30 bp. In some cases these include multiple distinct repeating units. Sometimes common CRISPR identification algorithms will flag these regions as CRISPR systems; however, closer inspection will reveal that the regions identified as spacer sequences are repeated in the array. The arrays are not immediately adjacent to the effectors, but they are in the same genomic region. SMART II system operons were generally devoid of adaptation module genes (e.g. genes involved in spacer acquisition).

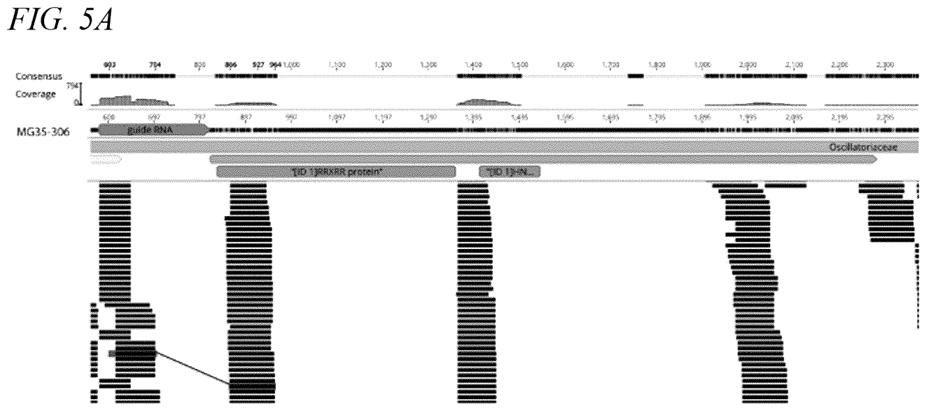

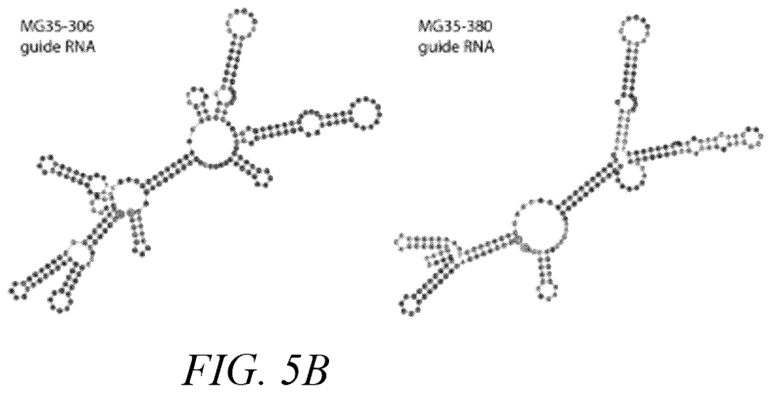

Environmental transcriptomic data for some SMART II systems confirmed in situ expression of CRISPR arrays and other repetitive regions in the natural environment. Transcription of the 5′ untranslated region (UTR) of some SMART II effectors was also observed from environmental expression data (FIG. 5), suggesting that this region may be important for either nuclease activity or regulation of the SMART system.

Preliminary in vitro experiments conducted with SMART II effector proteins, repetitive regions, and associated intergenic regions show that these enzymes have the ability to cleave dsDNA, possibly in a programmable manner. Results suggest that SMART II nuclease activity may be RNA or DNA guided, which may require using a repetitive region such as a CRISPR array, or via recognition of features encoded within the loci such as TIR or 5′ UTR. The 5′ UTR associated with SMART II effectors may encode an RNA guide for the effector to target DNA for cleavage activity.

Recently, short Cas9 homologs were reported to be programmable dsDNA nucleases using a guide RNA encoded in the 5′ UTR region of the effector (Altae-Tran, Kannan, et al. Science 2021). In these systems, a targeting “spacer” was identified upstream from the transcribed 5′ UTR of the effectors, suggesting that SMART II enzymes can be reprogrammed to target and cleave a specific DNA site by adding a “target spacer” to the 5′ end of predicted guide RNAs encoded in their 5′ UTR.

### Example 2—PAM Sequence Identification/Confirmation for the Endonucleases Described Herein (Prophetic)

Putative endonucleases are expressed in an E. coli lysate-based expression system (myTXTL, Arbor Biosciences). PAM sequences are determined by sequencing plasmids containing randomly-generated potential PAM sequences that can be cleaved by the putative nucleases. In this system, an E. coli codon optimized nucleotide sequence encoding the putative nuclease is transcribed and translated in vitro from a PCR fragment under control of a T7 promoter. A second PCR fragment with a minimal CRISPR array composed of a T7 promoter followed by a repeat-spacer-repeat sequence is transcribed in the same reaction. Successful expression of the endonuclease and repeat-spacer-repeat sequence in the TXTL system followed by CRISPR array processing provides active in vitro CRISPR nuclease complexes.

A library of target plasmids containing a spacer sequence matching that in the minimal array preceded by 8N mixed bases (potential PAM sequences) are incubated with the output of the TXTL reaction. After 1-3 hr, the reaction is stopped and the DNA is recovered via a DNA clean-up kit, e.g., Zymo DCC, AMPure XP beads, QiaQuick etc. Adapter sequences are blunt-end ligated to DNA with active PAM sequences that had been cleaved by the endonuclease, whereas DNA that had not been cleaved is now inaccessible for ligation. DNA segments comprising active PAM sequences are then amplified by PCR with primers specific to the library and the adapter sequence. The PCR amplification products are resolved on a gel to identify amplicons that correspond to cleavage events. The amplified segments of the cleavage reaction are also used as template for preparation of an NGS library or as a substrate for sanger sequencing. Sequencing this resulting library, which is a subset of the starting 8N library, reveals sequences with PAM activity compatible with the CRISPR complex. For PAM testing with a processed RNA construct, the same procedure is repeated except that an in vitro transcribed RNA is added along with the plasmid library and the minimal CRISPR array/tracr template is omitted. The following spacer sequence was used as a targets in these assays.

CGUGAGCCACCACGUCGCAAGCCUCGAC

### Example 3—Protocol for Predicted RNA Folding

Predicted RNA folding of the active single RNA sequence is computed at 37° using the method of Andronescu 2007. The color of the bases corresponds to the probability of base pairing of that base, where red is high probability and blue is low probability.

### Example 4—In Vitro Cleavage Efficiency (Prophetic)

Endonucleases are expressed as His-tagged fusion proteins from an inducible T7 promoter in a protease deficient E. coli B strain. Cells expressing the His-tagged proteins are lysed by sonication and the His-tagged proteins purified by Ni-NTA affinity chromatography on a HisTrap FF column (GE Lifescience) on an AKTA Avant FPLC (GE Lifescience). The eluate is resolved by SDS-PAGE on acrylamide gels (Bio-Rad) and stained with InstantBlue Ultrafast coomassie (Sigma-Aldrich). Purity is determined using densitometry of the protein band with ImageLab software (Bio-Rad). Purified endonucleases are dialyzed into a storage buffer composed of 50 mM Tris-HCl, 300 mM NaCl, 1 mM TCEP, 5% glycerol; pH 7.5 and stored at −80° C.

Target DNAs containing spacer sequences and PAM sequences (determined e.g., as in Example 2) are constructed by DNA synthesis. A single representative PAM is chosen for testing when the PAM has degenerate bases. The target DNAs are comprised of 2200 bp of linear DNA derived from a plasmid via PCR amplification with a PAM and spacer located 700 bp from one end. Successful cleavage results in fragments of 700 and 1500 bp. The target DNA, in vitro transcribed single RNA, and purified recombinant protein are combined in cleavage buffer (10 mM Tris, 100 mM NaCl, 10 mM MgCl₂) with an excess of protein and RNA and are incubated for 5 minutes to 3 hours, usually 1 hr. The reaction is stopped via addition of RNAse A and incubation at 60 minutes. The reaction is then resolved on a 1.2% TAE agarose gel and the fraction of cleaved target DNA is quantified in ImageLab software.

### Example 5—Activity in E. coli (Prophetic)

E. coli lacks the capacity to efficiently repair double-stranded DNA breaks. Thus, cleavage of genomic DNA can be a lethal event. Exploiting this phenomenon, endonuclease activity is tested in E. coli by recombinantly expressing an endonuclease and a guide RNA (determined e.g. as in Example 2) in a target strain with spacer/target and PAM sequences integrated into its genomic DNA.

For testing of nuclease activity in bacterial cells, BL21 (DE3) strains (NEB) are transformed with plasmids containing T7-driven effector and sgRNA (10 ng each plasmid), plated and grown overnight. The resulting colonies are cultured overnight in triplicate, then subcultured in SOB and grown to OD 0.4-0.6. 0.5 OD equivalent of cell culture is made chemocompetent according to standard kit protocol (Zymo Mix and Go kit) and transformed with 130 ng of a kanamycin plasmid either with or without a spacer and PAM in the backbone. After heat shock, transformations are recovered in SOC for 1 hr at 37° C., and nuclease efficiency is determined by a 5-fold dilution series grown on induction media (LB agar plates with antibiotics and 0.05 mM IPTG). Colonies are quantified from the dilution series to measure overall repression due to nuclease-driven plasmid cleavage.

Engineered strains with PAM sequences (determined e.g. as in Example 3) integrated into their genomic DNA are transformed with DNA encoding the endonuclease. Transformants were then made chemocompetent and are transformed with 50 ng of guide RNAs (e.g., crRNAs) either specific to the target sequence (“on target”), or non-specific to the target (“non target”). After heat shock, transformations were recovered in SOC for 2 hrs at 37U. Nuclease efficiency is then determined by a 5-fold dilution series grown on induction media. Colonies are quantified from the dilution series in triplicate.

### Example 6—(Prophetic) Testing of Genome Cleavage Activity of MG CRISPR Complexes in Mammalian Cells

To show targeting and cleavage activity in mammalian cells, the MG Cas effector protein sequences are tested in two mammalian expression vectors: (a) one with a C-terminal SV40 NLS and a 2A-GFP tag, and (b) one with no GFP tag and two SV40 NLS sequences, one on the N-terminus and one on the C-terminus. The NLS sequences comprise any of the NLS sequences described herein. In some instances, nucleotide sequences encoding the endonucleases are codon-optimized for expression in mammalian cells.

The corresponding crRNA sequence with targeting sequence attached is cloned into a second mammalian expression vector. The two plasmids are cotransfected into HEK293T cells. 72 hr after co-transfection of the expression plasmid and a gRNA targeting plasmid into HEK293T cells, the DNA is extracted and used for the preparation of an NGS-library. Percent NHEJ is measured via indels in the sequencing of the target site to demonstrate the targeting efficiency of the enzyme in mammalian cells. At least 10 different target sites are chosen to test each protein's activity.

### Example 7—Predicted Activity

In situ expression and protein sequence analyses indicate that the enzymes in SEQ ID NOs: 1 or 19-26 are active nucleases. They contains predicted endonuclease-associated domains (matching RRXRR and HNH_endonuclease Pfam domains) and contain predicted HNH and RuvC catalytic residues. Furthermore, the presence of an RRXRR motif, found in Ribonuclease H-like protein families, indicates potential RNA targeting or nuclease activity.

### Example 8—SMART II Guide RNA Prediction

The region comprising 400 bp immediately upstream from the start codon of SMART II effector sequences was extracted as potentially encoding a guide RNA required for activity (UTR). UTR sequences were aligned with MAFFT (mafft-ginsi algorithm) and regions showing blocks of conservation were annotated as putative guide RNAs.

### Example 9—Activity and PAM Determination Assays

The putative guide RNA predicted from RNASeq or from UTR alignment was folded in Geneious. A target spacer was appended to either the 5′ or 3′ end of the guide RNA to design a single guide RNA (sgRNA). The sgRNA was assembled via assembly PCR, purified with SPRI beads, and in vitro transcribed (IVT) following manufacturer's recommended protocol for short RNA transcripts (HiScribe T7 kit, NEB). RNA reactions were cleaned with the Monarch RNA kit and checked for purity via the Tapestation (Agilent).

Cleavage and PAM determination assays were performed with PURExpress (New England Biolabs). Briefly, the protein was codon optimized for E. coli and cloned into a vector with a T7 promoter and C-terminal His tag. The gene was PCR amplified with primer binding sites 150 bp upstream and downstream from the T7 promoter and terminator sequences, respectively. This PCR product was added to NEB PURExpress at 5 nM final concentration and expressed for 2 hr at 37° C. After this point, a cleavage reaction was assembled in 10 mM Tris pH 7.5, 100 mM NaCl, and 10 mM MgCl₂ with a 5-fold dilution of PURExpress, 5 nM of an 8N PAM plasmid library, and 50 nM of sgRNA targeting the PAM library.

The cleavage products from the PURExpress reactions were recovered via clean up with AMPure SPRI beads (Beckman Coulter). The DNA was blunted via addition of Klenow fragments and dNTPs (New England Biolabs). Blunt-end products were ligated with a 100-fold excess of double stranded adapter sequences and used as template for the preparation of an NGS library, from which PAM requirements were determined from sequence analysis.

Raw NGS reads were filtered by Phred quality score >20. The 24 bp representing the DNA sequence from the backbone adjacent to the PAM was used as a reference to find the PAM-proximal region and the 8 bp adjacent were identified as the putative PAM. The distance between the PAM and the ligated adapter was also measured for each read. Reads that did not have an exact match to the reference sequence or adapter sequence were excluded. PAM sequences were filtered by cut site frequency such that only PAMs with the most frequent cut site ±2 bp were included in the analysis. The filtered list of PAMs was used to generate a sequence logo using Logomaker.

### Example 10—Activity in Mammalian Cells with mRNA Delivery (Prophetic)

For genome editing using cell transfection/transformation with mRNA, the coding sequence is mouse or human codon optimized using algorithms from Twist Bioscience or Thermo Fisher Scientific (GeneArt). A cassette is constructed with two nuclear localization signals appended to the coding endonuclease sequence: SV40 and nucleoplasmin at the N and C terminal respectively. Additionally, untranslated regions from human complement 3 (C3) are appended to both the 5′ and 3′ to the coding sequence within the cassette.

This cassette is then cloned into a mRNA production vector upstream of a long poly A stretch. The mRNA construct organization can be as follows: 5′ UTR from C3—SV40 NLS—codon optimized SMART gene—nucleoplasmin NLS—3′ UTR from C3—107 polyA tail. Run-of transcription of the mRNA is then driven by a 17 promoter using an engineered 17 RNA polymerase (Hi-T: New England Biolabs). 5′ capping of the mRNA occurs co-transcriptionally using CleanCap AG (Trilink Biolabs). mRNA is then purified using MEGAclear Transcription Clean-Up kit (Thermo Fisher Scientific).

Mammalian cells are co-transfected with transcribed mRNA and a set of at least 10 guides targeting a genomic region of interest using Lipofectamine Messenger Max (Thermo Fisher Scientific). Cells are incubated for a period of time (e.g. 48 hours) followed by genomic DNA isolation using a Purelink Genomic DNA extraction kit (Fisher Scientific). The region of interest is amplified using specific primers. Editing is then assessed by Sanger sequencing using Inference of CRISPR Edits and NGS for a thorough analysis of edit outcomes.

TABLE 4
Listing of additional protein and nucleic acid sequences referred to
herein not included in the sequence listing

| | SEQ | | | | Other | |
|---|---|---|---|---|---|---|
| Category | ID: | Description | Type | Organism | Information | Sequence |
| MG35 | 29 | MG35-306 | nucleotide | unknown | uncultivated | AGTCAGCACCCCGCTCT |
| putative | | effector | | | organism | AAAGAGACGGGGCTTCA |
| single | | putative | | | | TGCCTCGTTGAATTAGG |
| guide | | single guide | | | | TAGCTGACCCGGCTAAG |
| RNA | | RNA | | | | TCTTTAGAGGACTACGTT |
| encoding | | encoding | | | | TTTTAAGTCATAACACCT |
| sequences | | sequence | | | | ACGAATGCTTCACCAGT |
| | | | | | | TTGTAGCTCTGTTGTTAA |
| | | | | | | TCGTTAAACAATCCTACG |
| | | | | | | AGGGGTAAGATAGTGCG |
| | | | | | | GTTAATATAAAAAGCTTA |
| | | | | | | AAAAACATTGCCAAGGT |
| | | | | | | GAAGATTACCTCAGAAA |
| | | | | | | TGAGAGTTTTTTTAA |
| MG35 | 30 | MG35-380, | nucleotide | unknown | uncultivated | GTCAACTACCCCGCCCT |
| putative | | MG35-381, | | | organism | GAAAGGCGGAGCTTGTT |
| single | | MG35-382, | | | | GAAAGACAAGCTGGGTT |
| guide | | MG35-383 | | | | GGCCAGGGAGAGAAAGG |
| RNA | | effectors | | | | TGTTGAAAGACGCCAAT |
| encoding | | putative | | | | CAACGTGTGCAACAGGT |
| sequences | | single guide | | | | CGTCAAGACGCACCGGC |
| | | RNA | | | | GAATGCTTCCTCAGTTC |
| | | encoding | | | | GCCGCTCTGCAAGGCGG |
| | | sequence | | | | GAATCATGCTGGCGAAA |
| | | | | | | GGTAAAGCGCCGAAGGT |
| | | | | | | TCTCACCGCTGCCGCAA |
| | | | | | | GGCAGGAGTCGGTTGCG |
| | | | | | | CACAGTCCCGAGGGGAC |
| | | | | | | GCAAGGCCCCGTCACAA |
| | | | | | | GGCCCGTAAGGGCA |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-81. (canceled)

82. An engineered nuclease system comprising:
a) an endonuclease comprising a RuvC domain and an HNH domain; and
b) an engineered guide ribonucleic acid structure capable of forming a complex with said endonuclease comprising:
i) a guide ribonucleic acid sequence capable of hybridizing to a target deoxyribonucleic acid sequence; and
ii) a ribonucleic acid sequence capable of binding to said endonuclease, wherein said endonuclease comprises a sequence having at least 70% sequence identity to any one of SEQ ID NOs: 1 or 19-28.

83. The engineered nuclease system of claim 82, wherein said endonuclease is an archaeal endonuclease.

84. The engineered nuclease system of claim 82, wherein said endonuclease is a class 2, type II Cas endonuclease.

85. The engineered nuclease system of claim 82, wherein said endonuclease further comprises one or more of: an arginine-rich region comprising an RRxRR motif, a domain with PF14239 homology, a recognition (REC) domain, a bridge helix (BH) domain, a wedge (WED) domain, or a PAM interacting (PI) domain.

86. The engineered nuclease system of claim 85, wherein said arginine-rich region, said domain with PF14239 homology, said recognition (REC) domain, said bridge helix (BH) domain, said wedge (WED) domain, or said PAM interacting (PI) domain comprises a sequence having at least 85% sequence identity to an arginine-rich region comprising an RRxRR motif, a domain with PF14239 homology, a recognition (REC) domain, a bridge helix (BH) domain, a wedge (WED) domain, or a PAM interacting (PI) domain, respectively, of any one of SEQ ID NOs: 1, or 19-28.

87. The engineered nuclease system of claim 82, wherein said endonuclease comprises a sequence having less than 80% sequence identity to a SpCas9 endonuclease.

88. The engineered nuclease system of claim 82, wherein said endonuclease comprises one or more nuclear localization sequences (NLSs) proximal to an N-terminus or a C-terminus of said endonuclease.

89. The engineered nuclease system of claim 88, wherein said NLS comprises a sequence selected from any one of SEQ ID NOs: 2-18.

90. The engineered nuclease system of claim 82, wherein said ribonucleic acid sequence comprises a sequence having at least 90% sequence identity to any one of SEQ ID NOs: 29 or 30.

91. The engineered nuclease system of claim 82, wherein said sequence identity is determined by aBLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

92. The engineered nuclease system of claim 82, further comprising a single- or double-stranded deoxyribonucleic acid repair template comprising from 5′ to 3′: a first homology arm comprising a sequence of at least 20 nucleotides 5′ to said target deoxyribonucleic acid sequence, a synthetic deoxyribonucleic acid sequence of at least 10 nucleotides, and a second homology arm comprising a sequence of at least 20 nucleotides 3′ to said target deoxyribonucleic acid sequence.

93. The engineered nuclease system of claim 82, wherein said guide ribonucleic acid sequence is complementary to a eukaryotic, a fungal, a plant, a mammalian, or a human genomic sequence.

94. The engineered nuclease system of claim 82, wherein said guide ribonucleic acid sequence is 15-24 nucleotides in length.

95. The engineered nuclease system of claim 82, wherein said engineered guide ribonucleic acid structure comprises a single ribonucleic acid polynucleotide comprising said guide ribonucleic acid sequence and said ribonucleic acid sequence.

96. A method of modifying a target nucleic acid locus, said method comprising contacting said target nucleic acid locus with:
a) an endonuclease comprising a RuvC domain and an HNH domain, wherein said endonuclease comprises a sequence having at least 70% sequence identity to any one of SEQ ID NOs: 1 or 19-28; and
b) an engineered guide ribonucleic acid structure configured to form a complex with said endonuclease, wherein said engineered guide ribonucleic acid structure comprises:
i) a guide ribonucleic acid sequence configured to hybridize to a portion of said target nucleic acid locus; and
ii) a ribonucleic acid sequence configured to bind to said endonuclease, wherein said complex modifies said target nucleic acid locus.

97. The method of claim 96, wherein said ribonucleic acid sequence comprises a sequence having at least 90% sequence identity to any one of SEQ ID NOs: 29 or 30.

98. The method of claim 96, wherein said modifying comprises binding, nicking, cleaving, or marking said target nucleic acid locus.

99. The method of claim 96, wherein said target nucleic acid locus comprises deoxyribonucleic acid (DNA) or ribonucleic acid (RNA).

100. The method of claim 96, wherein said target nucleic acid locus is within a cell.

101. The method of claim 100, wherein said cell is a eukaryotic cell, an animal cell, a mammalian cell, a rodent cell, a primate cell, or a human cell.
